(19) United States
(12) Patent Application Publication
(10) Pub. No.: US 2022/0018647 A1
(43) Pub. Date: Jan. 20, 2022

(54) SENSOR BODY AND SENSOR DEVICE

(71) Applicant: BANDO CHEMICAL INDUSTRIES, LTD., Hyogo (JP)

(72) Inventors: Hideo OTAKA, Hyogo (JP); Mikio TSUTSUMI, Hyogo (JP)

(73) Assignee: BANDO CHEMICAL INDUSTRIES, LTD., Hyogo (JP)

(21) Appl. No.: 17/296,015

(22) PCT Filed: Nov. 26, 2019
(86) PCT No.: PCT/JP2019/046213

§ 371 Date: May 21, 2021

(30) Foreign Application Priority Data
Nov. 28, 2018 (JP) 2018-222504

(51) Int. Cl. G01B 7/30; G01B 7/02

(52) U.S. Cl. CPC G01B 7/30; G01B 7/02

(57) ABSTRACT

A sensor body including: a plate-like flexible intermediate layer that is stretchable in a surface direction; a first sensor element laminated on one surface of the intermediate layer; and a second sensor element laminated on the other surface of the intermediate layer facing the one surface. The first sensor element is configured to be capable of detecting a length corresponding to a length of a first reference line segment on the one surface of the intermediate layer, and the second sensor element is configured to be capable of detecting a length corresponding to a length of a second reference line segment that is on the other surface of the intermediate layer and parallel to the first reference line segment.

### TECHNICAL FIELD

The present invention relates to a sensor body and a sensor device provided with the sensor body.

### BACKGROUND ART

As a method for measuring a bending angle of a joint of a human body or the like, various methods have been proposed.

For example, Patent Literature 1 proposes a joint angle sensor that is worn on a joint part of a human body and detects the bending angle of the joint. The joint angle sensor includes: an elastically deformable capacitive sensor in which electrode films made of a conductive elastomer are provided on each surface of a dielectric film made of an elastomer; and a deformation regulation member that is superposed on one electrode film side of the capacitive sensor and regulates the shape of the bending deformation of the capacitive sensor.

The joint angle sensor described in Patent Literature 1 includes a deformation regulation member made of polyimide, polyethylene, spring steel, or the like. The joint angle sensor deforms the capacitive sensor, while limiting the deformation of the capacitive sensor to a specific deformation form by using the deformation regulation member, to measure the joint angle of the human body.

According to Patent Literature 1, the use of the joint angle sensor makes it possible to detect the bending angle of the joint of the human body with high accuracy and excellent responsiveness while avoiding a wearer's feeling of discomfort without limiting the movement of the joint of the human body.

### CITATION LIST

### Patent Literature

Patent Literature 1: Japanese Unexamined Patent Publication No. 2015-217127

### SUMMARY OF INVENTION

### Technical Problem

The joint angle sensor described in Patent Literature 1 includes the deformation regulation member. The deformation regulation member has the rigidity required to limit the deformation form of the capacitive sensor. Thus, when the bending angle of the joint is measured by the joint angle sensor, there is a possibility that the movement of the joint is limited by the deformation regulation member. Further, in order to measure the bending angle of the joint by using the joint angle sensor without limiting the movement of the joint, the attachment place and the attachment method for the joint angle sensor are limited significantly.

The joint angle sensor disclosed in Patent Literature 1 measures the joint angle by deforming the capacitive sensor so as to match a predetermined operational model. The operational model assumes a joint in which degrees of freedom can be regarded as one axis, such as a knee joint. Hence the joint angle sensor has not been suitable for measuring a bending angle of a joint having two or more axes of degrees of freedom, such as a wrist joint.

The reason for this is that when the joint angle sensor described in Patent Document 1 is used to measure the bending angle of the joint having two or more axes of degrees of freedom, the deformation of the capacitive sensor is regulated in accordance with the bending of the joint of one axis, so that the free bending of the joint of the other axis is inhibited.

### Solution to Problem

Under such circumstances, the inventors of the present invention have diligently studied to provide a sensor device capable of accurately measuring a bending angle of a joint having two or more axes of degrees of freedom and have completed a new sensor body and sensor device.

(1) A sensor body of the present invention is provided with: a plate-like flexible intermediate layer that is stretchable in a surface direction; a first sensor element laminated on one surface of the intermediate layer; and a second sensor element laminated on the other surface of the intermediate layer facing the one surface. The first sensor element is configured to be capable of detecting a length corresponding to a length of a first reference line segment on the one surface of the intermediate layer, and the second sensor element is configured to be capable of detecting a length corresponding to a length of a second reference line segment that is on the other surface of the intermediate layer and parallel to the first reference line segment.

According to the sensor body, the sensor elements (first sensor element and second sensor element) are provided on both surfaces of the plate-like intermediate layer, respectively. Thus, with these sensor elements, it is possible to measure changes in the length corresponding to the length of the first reference line segment and the length corresponding to the length of the second reference line segment set on both surfaces of the intermediate layer.

By using the measurement results of the change in the length corresponding to the length of the first reference line segment and the change in the length corresponding to the length of the second reference line segment, the sensor body can calculate the bending angle of the sensor body based on the difference between the amounts of change in the length corresponding to the length of the first reference line segment and the amounts of change in the length corresponding to the length of the second reference line segment and can calculate the amount of elongation of the sensor body based on the amounts of change in the average length of the length corresponding to the length of the first reference line segment and the length corresponding to the length of the second reference line segment.

Thus, when the sensor body is placed on a measuring object and used, the bending angle and the amount of elongation of the measuring object can be measured. Specific methods for calculating these will be described later.

Hereinafter, in the present specification, when simply “the length of the first reference line segment” is referred to, “the length of the first reference line segment” is a concept including “the length corresponding to the length of the first reference line segment”, and when simply “the length of the second reference line segment” is referred to, “the length of the second reference line segment” is a concept including “the length corresponding to the length of the second reference line segment”.

(2) In the sensor body, preferably, the first sensor element and the second sensor element are both capacitive sensor elements.

Each of the capacitive sensor element is preferably a sensor element including a dielectric layer made of an elastomer, a first electrode layer formed on the upper surface of the dielectric layer, and a second electrode layer formed on the lower surface of the dielectric layer. Each of the sensor element has a part in which the first electrode layer and the second electrode layer face each other and that serves as a detection portion. A capacitance of the detection portion changes in accordance with deformation of the dielectric layer.

Such a capacitive sensor element is suitable for measuring the change in the length corresponding to the length of the first reference line segment and the change in the length corresponding to the length of the second reference line segment, which occur at the time of deformation of the sensor body, without inhibiting the deformation of the sensor body. Therefore, the sensor body provided with the capacitive sensor element is particularly suitable for measuring a bending angle and an amount of elongation of a measuring object at the time of deformation thereof.

(3) It is preferable that the sensor body be further provided with a plate-like attachment member having the same thickness as the intermediate layer and higher rigidity than the intermediate layer, and the attachment member be provided outside each end of the first reference line segment of the intermediate layer.

The sensor body having such a configuration can more accurately measure the bending angle and the amount of elongation of the measuring object at the time of deformation thereof.

Further, the sensor body is particularly suitable for being placed on a measuring object by fixing the attachment member to the measuring object and for measuring the bending angle of the measuring object.

(4) The sensor body is preferably used for measuring a joint angle of a human body.

The human body has many joints having a two-axis degree of freedom (or two or more axes of degrees of freedom) and is thus particularly suitable as the measuring object of the sensor body.

(5) A sensor device of the present invention includes the sensor body and an analyzer, and the analyzer measures a change in the length corresponding to the length of the first reference line segment and a change in the length corresponding to the length of the second reference line segment at a time of deformation of the sensor body and calculates a bending angle and amount of elongation of a measuring object based on the obtained measurement results.

Because including the sensor body, the sensor device can measure one of or both the bending angle and the amount of elongation of the measuring object at the time of deformation thereof and can simultaneously measure the bending angle and the amount of elongation.

### Advantageous Effects of Invention

According to the present invention, the amount of elongation and/or the bending angle of the measuring object can be measured easily and accurately.

### BRIEF DESCRIPTION OF DRAWINGS

FIG. 1 is a schematic diagram showing a sensor device according to an embodiment of the present invention.

FIG. 2A is a perspective view showing an example of a sensor element provided in a sensor body according to the embodiment of the present invention, and FIG. 2B is a cross-sectional view taken along line A-A in FIG. 2A.

FIG. 3A is an exploded perspective view showing the sensor body according to the embodiment of the present invention, and FIG. 3B is a cross-sectional view taken along line B-B of the sensor body shown in FIG. 3A.

FIGS. 4A to 4C are schematic views for explaining a method for grasping a deformed state of a sensor body 2 according to the present embodiment.

FIGS. 5A to 5C are views for explaining an example of the usage of the sensor body according to the embodiment of the present invention.

FIG. 6 is a view for explaining another example of the usage of the sensor body according to the embodiment of the present invention.

FIG. 7 is a view for explaining another example of the usage of the sensor body according to the embodiment of the present invention.

FIG. 8A is a view for explaining another example of the usage of the sensor body according to the embodiment of the present invention, and FIG. 8B is a view for explaining characteristics of the sensor body according to the embodiment of the present invention.

FIG. 9 is a view for explaining an example of a detection circuit provided in an analyzer of the sensor device according to the embodiment of the present invention.

FIG. 10A is a perspective view showing another example of the sensor body according to the embodiment of the present invention, and FIG. 10B is a cross-sectional view taken along line C-C of FIG. 10A.

FIG. 11A is a perspective view showing another example of the sensor element provided in the sensor body according to the embodiment of the present invention, and FIG. 11B is a cross-sectional view taken along line D-D in FIG. 11A.

FIG. 12 is a cross-sectional view showing another example of the sensor body according to the embodiment of the present invention.

FIG. 13A is a plan view schematically showing a sensor body produced in the present example, and FIG. 13B is a cross-sectional view taken along line E-E in FIG. 13A.

FIG. 14 is a photograph showing a measurement jig to which a sensor body has been attached.

FIGS. 15A and 15B are views for explaining bending evaluation 1.

FIG. 16 is a graph showing the results of the bending evaluation 1 performed in the example.

FIG. 17 is a graph showing results of bending evaluation 2 performed in the example.

FIG. 18 is a graph showing results of elongation evaluation performed in the example.

### DESCRIPTION OF EMBODIMENTS

Embodiments of the present invention will be described below with reference to the drawings.

(First Embodiment)

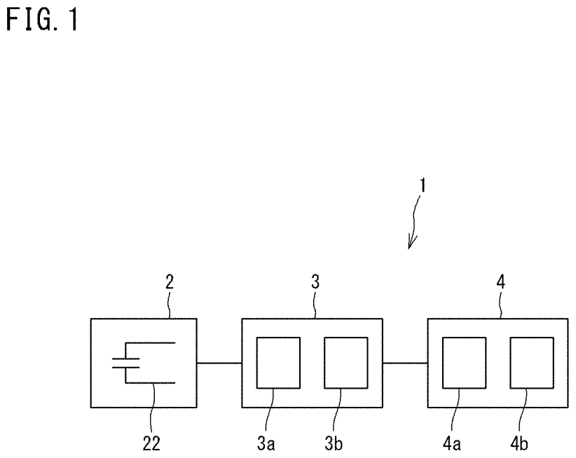

FIG. 1 is a schematic diagram showing a sensor device according to an embodiment of the present invention.

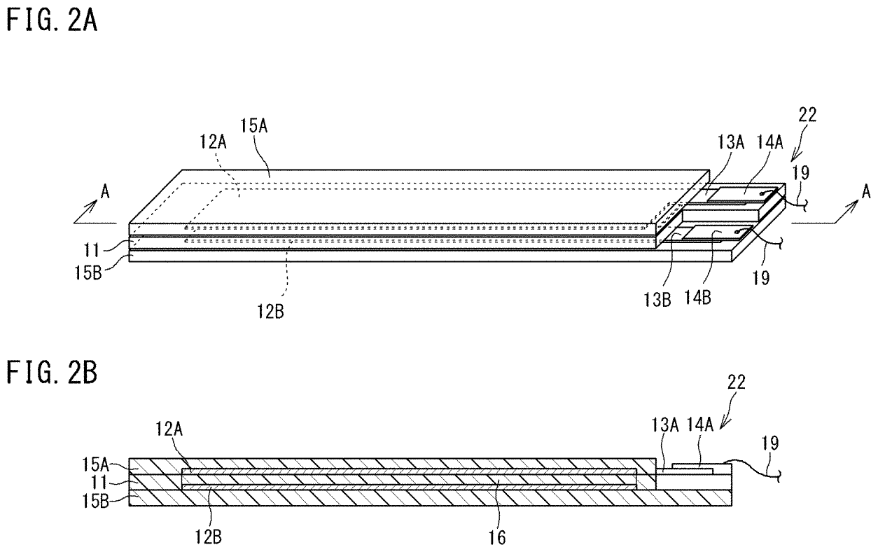

FIG. 2A is a perspective view showing an example of a sensor element provided in a sensor body according to the embodiment of the present invention, and FIG. 2B is a cross-sectional view taken along line A-A in FIG. 2A.

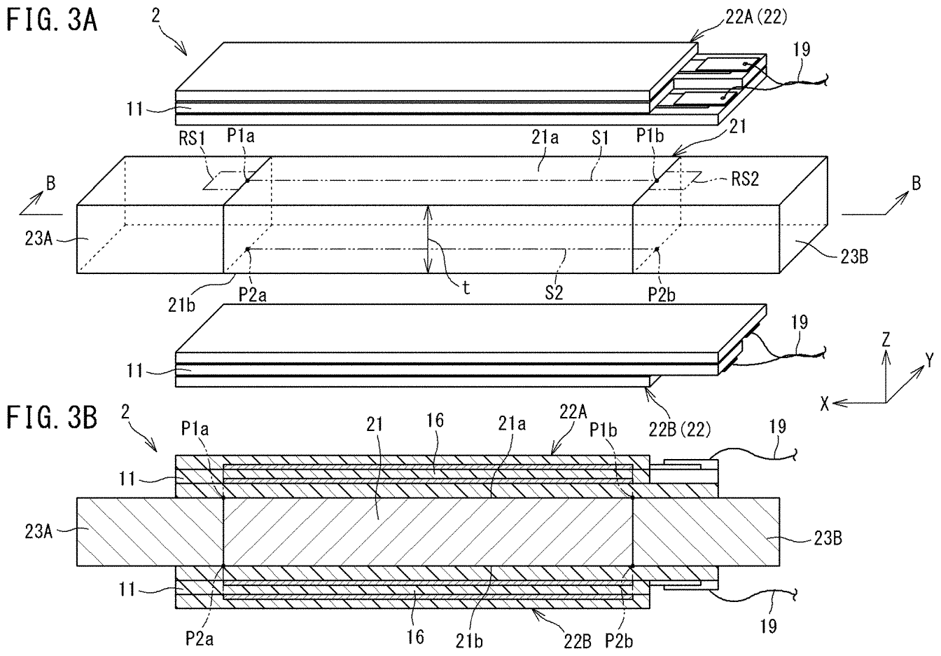

FIG. 3A is an exploded perspective view showing the sensor body according to the embodiment of the present invention, and FIG. 3B is a cross-sectional view taken along line B-B of the sensor body shown in FIG. 3A.

As shown in FIG. 1, the sensor device 1 according to the present embodiment is provided with a sensor body 2 including a capacitive sensor element 22 (cf. FIGS. 2A and 2B), an analyzer 3 electrically connected to the sensor body 2, and an indicator 4 for indicating results of measurement in the analyzer 3.

As shown in FIGS. 3A and 3B, the sensor body 2 is provided with a plate-like intermediate layer 21, two sensor elements 22 (first sensor element 22A and second sensor element 22B) laminated on both surfaces (the upper surface and lower surface) of the intermediate layer 21, respectively, and attachment members 23A, 23B formed of plate-like bodies having the same thickness as the intermediate layer 21 and provided to sandwich the intermediate layer 21 in the longitudinal direction.

In the description of the embodiment of the present invention, as shown in FIG. 3A, the longitudinal direction of the intermediate layer 21 (the horizontal direction in FIG. 3A) is referred to as the X-direction, the thickness direction of the intermediate layer 21 (the vertical direction in FIG. 3A) is referred to as the Z-direction, and the direction perpendicular to the X-direction and the Z-direction is referred to as the Y-direction.

The intermediate layer 21 is a plate-like body made of an elastomer composition or the like. The intermediate layer 21 is a member that can be freely deformed in such a manner as to be stretched, curved, or bent in accordance with an external force when the external force is applied, while maintaining its shape in a natural state.

The first sensor element 22A and the second sensor element 22B are laminated on both surfaces of the intermediate layer 21 via an adhesive layer (not shown).

The first sensor element 22A and the second sensor element 22B are the same sensor elements.

The adhesive layer for fixing the first sensor element 22A and the second sensor element 22B to the intermediate layer is a flexible adhesive layer that does not inhibit the deformation of the intermediate layer 21 and the sensor elements 22A, 22B.

The attachment members 23A, 23B are fixed outside both ends of the intermediate layer 21 in the longitudinal direction (X-direction) via adhesive layers (not shown).

The attachment members 23A, 23B are members having the same thickness as the intermediate layer 21.

The attachment members 23A, 23B are members made of polyethylene terephthalate (PET) or the like and sufficiently higher in rigidity than the intermediate layer 21. Preferably, the attachment members 23A, 23B are members that are not substantially deformed under the condition of use. The material of the attachment members 23A, 23B is not limited to PET, but the attachment members 23A, 23B may be made of another resin having sufficient rigidity or may be made of metal, wood, or the like.

As shown in FIGS. 2A and 2B, the sensor element 22 includes a sheet-like dielectric layer 11 made of an elastomer composition, a top electrode layer (first electrode layer) 12A formed on the top surface of the dielectric layer 11, a bottom electrode layer (second electrode layer) 12B formed on the bottom surface of the dielectric layer 11, a top conducting wire 13A coupled to the top electrode layer 12A, a bottom conducting wire 13B coupled to the bottom electrode layer 12B, a top connecting portion 14A attached to the end of the top conducting wire 13A on the opposite side to the top electrode layer 12A, a bottom connecting portion 14B attached to the end of the bottom conducting wire 13B on the opposite side to the bottom electrode layer 12B, and a top protective layer 15A and a bottom protective layer 15B respectively laminated on the top said and bottom side of the dielectric layer 11.

A lead wire 19 is connected to each of the top connecting portion 14A and the bottom connecting portion 14B of the sensor element 22, and the sensor element 22 is connected to the analyzer 3 (cf. FIG. 1) via the lead wire 19.

The top electrode layer 12A and the bottom electrode layer 12B have the same shape in a plan view, and the top electrode layer 12A and the bottom electrode layer 12B entirely face each other across the dielectric layer 11. In the sensor element 22, a part where the top electrode layer 12A and the bottom electrode layer 12B face each other is taken as a detection portion 16.

In the embodiment of the present invention, the top electrode layer 12A and the bottom electrode layer 12B provided in the capacitive sensor element do not necessarily have to face each other entirely with the dielectric layer interposed therebetween, and at least a part thereof may face each other.

In the sensor element 22, the dielectric layer 11 is made of an elastomer composition. Hence the sensor element 22 can be deformed in such a manner as to be stretched, bent, or curved in the surface direction and the thickness direction.

When the dielectric layer 11 is deformed, the capacitance of the detection portion 16 of the sensor element 22 changes in correlation with the amount of deformation of the dielectric layer 11 (a change in the area of the electrode layer).

Therefore, the amount of deformation of the sensor element 22 can be detected by detecting the change in the capacitance of the detection portion.

The sensor element 22 is provided on each of the surfaces (upper surface and lower surface) of the intermediate layer 21 in the sensor body 2.

The sensor element 22 is laminated on the intermediate layer 21 such that both end edges in the X-direction of the detection portion 16 of the sensor element 22 overlap with both end edges in the X-direction of the intermediate layer 21 in the thickness direction (Z-direction) of the sensor body.

In the sensor body 2, a line segment connecting respective midpoint P1a, P1b of both end edges in the X-direction on an upper surface 21a of the intermediate layer 21 is a first reference line segment S1, and a line segment connecting respective midpoint P2a, P2b of both end edges in the X-direction on a lower surface 21b of the intermediate layer 21 is a second reference line segment S2.

Thus, it can also be said that in the sensor body 2, the sensor elements 22 are provided on both surfaces of the intermediate layer 21 such that the first reference line segment S1 and the second reference line segment S2 set on the upper and lower surfaces of the intermediate layer 21 and the detection portion 16 of the sensor element 22 overlap with each other in the thickness direction.

In the sensor body 2, when the intermediate layer 21 of the sensor body 2 is deformed, the sensor elements 22 measures the amounts of change in the respective lengths of the first reference line segment S1 and the second reference line segment S2.

When the intermediate layer 21 is deformed and the lengths of the first reference line segment S1 and the second reference line segment S2 change, the detection portion 16 of the sensor element 22 is deformed following the deformation, and as a result, the capacitance of the detection portion 16 changes. Therefore, the amounts of change in the lengths of the first reference line segment S1 and the second reference line segment S2 can be calculated based on the amount of change in the capacitance of the detection portion.

In the sensor body 2 according to the present embodiment, the length corresponding to the length of the first reference line segment S1 is the length in the longitudinal direction (X-direction in FIG. 3) of the detection portion 16 provided in the sensor element 22A, and the length corresponding to the length of the second reference line segment S2 is the length in the longitudinal direction (X-direction in FIG. 3) of the detection portion 16 provided in the sensor element 22B.

The sensor body 2 can obtain the deformed state (bending angle and amount of elongation) of the sensor body 2 based on the amounts of change in the lengths of the first reference line segment S1 and the second reference line segment S2.

Specifically, the sensor body 2 can grasp a change in the length of the central part of the intermediate layer 21, which occurs at the time of deformation of the intermediate layer 21, as an amount of elongation and can grasp the magnitude of an angle formed by the first reference plane RS1 and the second reference plane RS2 as a bending angle.

Therefore, the sensor body 2 can measure an amount of elongation and a bending angle of a measuring object, which is subjected to elastic deformation and bending deformation in a predetermined form, by being placed on the measuring object and measuring a change in the above-described length and a change in the above-described magnitude of the angle which occur at the time of deformation of the intermediate layer 21.

The sensor body 2 includes an adhesive layer (not shown) configured to place the sensor body 2 on the measuring object on the lower surface side, and the sensor body 2 can be stuck to the measuring object by the adhesive layer.

In the embodiment of the present invention, the sensor body may not necessarily include the adhesive layer.

In the sensor body 2, the length of a line segment which is equidistant from the first reference line segment S1 and the second reference line segment S2 in the thickness direction of the intermediate layer 21 (the Z-direction in FIG. 3) is taken as the length of the central part of the intermediate layer 21.

In the sensor body 2, an arbitrary region, which is in the same plane as the upper surface 21a of the intermediate layer 21, includes the end point P1a of the first reference line segment S1, and is outside the end point P1a (opposite to the first reference line segment S1 side), is taken as a first reference plane (RS1 in FIG. 3A), and an arbitrary region, which is in the same plane as the upper surface 21a of the intermediate layer 21, includes the end point P1b of the first reference line segment S1, and is outside the end point P1b, is taken as a second reference plane (RS2 in FIG. 3A). Thus, in the sensor body 2, the upper surface of the attachment member 23A corresponds to the first reference plane RS1, and the upper surface of the attachment member 23B corresponds to the second reference plane RS2.

The sensor body 2 calculates the bending angle of the sensor body 2 as the angle formed by the first reference plane RS1 and the second reference plane RS2.

A method for measuring the amount of elongation and the bending angle of the measuring object by the sensor body 2 will be described with reference to the drawings.

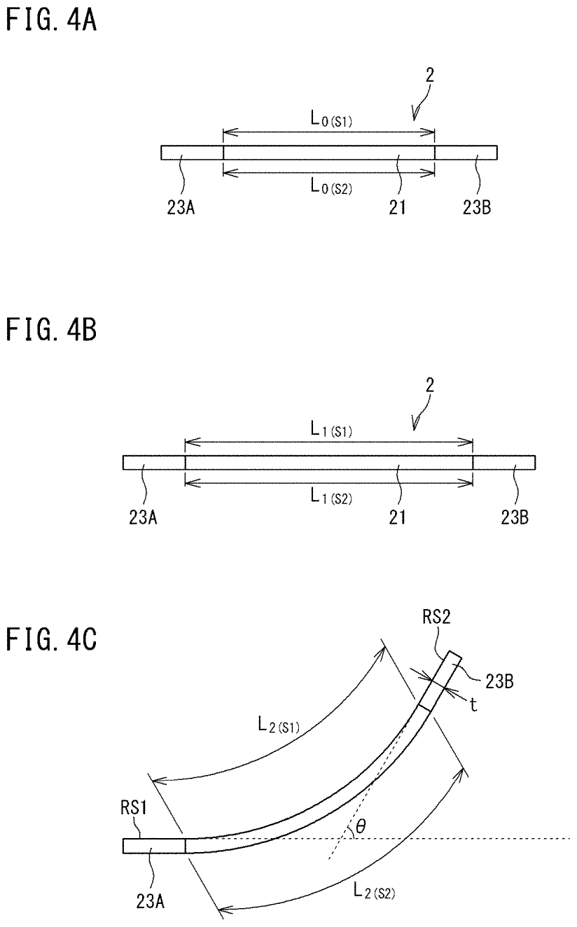

FIGS. 4A to 4C are schematic views for explaining a method for grasping the deformed state of the sensor body 2 according to the present embodiment. In FIGS. 4A to 4C, the first sensor element 22A and the second sensor element 22B are omitted in order to simplify the drawings.

When the sensor body 2 is deformed from an initial state shown in FIG. 4A to a state shown in FIG. 4B, the intermediate layer 21 of the sensor body 2 is elongated in the surface direction.

In this case, the length of the first reference line segment S1 changes from L_{0(S1)} to L_{1(S1)}, and the length of the second reference line segment S2 changes from L_{0(S2)} to L_{1(S2)}. At this time, the amount of change in the length of the first reference line segment S1 can be detected by the amount of deformation of the first sensor element 22A, and the amount of change in the length of the second reference line segment S2 can be detected by the amount of deformation of the second sensor element 22B. This is because when the first sensor element 22A is deformed following the deformation of the upper surface 21a of the intermediate layer 21, the capacitance of the detection portion 16 of the first sensor element 22A changes, and when the second sensor element 22B is deformed following the deformation of the lower surface 21b of the intermediate layer 21, the capacitance of the detection portion 16 of the second sensor element 22B changes.

Then, the amount of change (L_{1(S1)}-L_{0(S1)}) in the length of the first reference line segment S1 is compared with the amount of change (L_{1(S2)}-L_{0(S2)}) in the length of the second reference line segment S2. In this case, the difference between the amounts of change in the two line segments is 0. From this fact, it can be understood that only the elastic deformation has occurred in the sensor body 2 and that the bending deformation has not occurred.

Further, an average value between the amount of change in the length of the first reference line segment S1 and the amount of change in the length of the second reference line segment S2 (the same value as each of the amount of change in the length of the first reference line segment S1 and the amount of change in the length of the second reference line segment S2) is taken as the amount of elongation of the sensor body 2.

On the other hand, when the sensor body 2 is deformed from the initial state shown in FIG. 4A to a state shown in FIG. 4C, the intermediate layer 21 of the sensor body 2 is deformed by bending.

In this case, the length of the first reference line segment S1 changes from L_{0(S1)} to L_{2(S1)}, and the length of the second reference line segment S2 changes from L_{0(S2)} to L_{2(S2)}. At this time as well, the amount of change in the length of the first reference line segment S1 can be detected by the amount of deformation of the first sensor element 22A, and the amount of change in the length of the second reference line segment S2 can be detected by the amount of deformation of the second sensor element 22B.

In the sensor body 2, when only the bending deformation occurs in the intermediate layer 21 and no elastic deformation occurs, one of the first reference line segment S1 and the second reference line segment S2 is elongated, and the other is contracted. In addition, when only the bending deformation occurs, the total value of the length of the first reference line segment S1 and the length of the second reference line segment S2 remains constant before and after the bending deformation.

In the example shown in FIG. 4C, the sensor body 2 after the deformation is in the state of

(1) L_{2(S1)}<L_{0(S1)}, and L_{2(S2)}>L_{0(S2)}. It is thereby possible to understand the fact that the sensor body 2 has been deformed by bending and to grasp the direction of the bending.

The sensor body 2 is also in the state of

(2) L_{0(S1)}+L_{0(S2)}=L_{2(S1)}+L_{2(S2)}. It is thereby possible to understand that the sensor body 2 has not been elastically deformed and that the deformation having occurred in the sensor body 2 is only the bending deformation.

In the sensor body 2 before and after the deformation, when L_{0(S1)}+L_{0(S2)}≠L_{2(S1)}+L_{2(S2)}, it means that the sensor body 2 has at least been elongated or stretched.

Then, the amount of change (L_{2(S1)}−L_{0(S1)}) in the length of the first reference line segment S1 and the amount of change (L_{2(S2)}−L_{0(S2)}) in the length of the second reference line segment S2 are each calculated, so that the bending angle of the sensor body 2 can be grasped.

Specifically, the angle formed by the first reference plane RS1 and the second reference plane RS2 as the bending angle can be obtained using Equations (1) to (3) below:

Assuming that the angle formed by the first reference plane RS1 and the second reference plane RS2 is θ (rad) and that the thickness of the intermediate layer 21 of the sensor body 2 is t, the relationship between the length L_{2(S1)} of the first reference line segment S1 and the length L_{2(S2)} of the second reference line segment S2 is expressed by:

L_{2(S2)}=L_{2(S1)}+θt (1),

and
the difference between the length of the first reference line segment S1 and the length of the second reference line segment S2 is expressed by:

L_{2(S2)}−L_{2(S1)}=θt (2)

Therefore, the above-mentioned θ is obtained by:

θ(rad)=(L_{2(S2)}−L_{2(S1)})/t (3)

Therefore, the bending angle of the sensor body 2 can be calculated based on the length L_{2(S1)} of the first reference line segment S1 and the length L_{2(S2)} of the second reference line segment S2.

Further, since the sensor body according to the embodiment of the present invention calculates the bending angle based on the lengths of the two reference line segments, the bending angle can be accurately measured without performing calibration.

When the intermediate layer 21 is elongated and is deformed by bending at the same time, the sensor body 2 can individually calculate the amount of elongation and the bending angle.

As described above, when the sensor body 2 is deformed by bending, the sensor body 2 is deformed such that one of the first reference line segment S1 and the second reference line segment S2 is elongated and the other is contracted, and the sum of the respective lengths of the first reference line segment S1 and the second reference line segment S2 is kept constant.

When the sensor body 2 is stretched, the amounts of elongation of the first reference line segment S1 and the second reference line segment S2 are the same.

Therefore, when the sensor body 2 (intermediate layer 21) is deformed, it is first determined which is the deformed state of the sensor body 2 among:
(a) only bending deformation;
(b) only elastic deformation, and
(c) composite deformation of bending deformation and elastic deformation,
based on the respective amounts of deformation of the first reference line segment S1 and the second reference line segment S2. When the deformed state of the sensor body 2 is (a) only bending deformation or (b) only elastic deformation, the bending angle or the amount of elongation may be obtained by the method described above.

On the other hand, when the deformed state of the sensor body 2 (intermediate layer 21) is determined to be the composite deformation described in (c) above, the bending angle and the amount of elongation are each obtained.

First, the average value of the amount of change in the length of the first reference line segment S1 and the amount of change in the length of the second reference line segment S2 is calculated in the same manner as the above-described method for obtaining the amount of elongation. As described above, this value is taken as the amount of elongation of the sensor body 2.

Next, the difference between the respective lengths of the first reference line segment S1 and the second reference line segment S2 after the deformation and the average value of the lengths of the first reference line segment S1 and the second reference line segment S2 after the deformation is calculated, and the portion longer than the average value is taken as the amount of elongation while the portion shorter than the average value is taken as the amount of contraction. The bending angle is calculated using the above values by the calculation method described above.

As thus described, when the intermediate layer 21 is elastically deformed and is deformed by bending at the same time, the sensor body 2 can individually calculate the amount of elongation and the bending angle. Therefore, the deformed state of the measuring object can be grasped by using the sensor body 2.

The intermediate layer 21 of the sensor body 2 can be subjected to the composite deformation. The thickness of the intermediate layer 21 changes in accordance with elongation and contraction.

On the other hand, the above-described calculation method for the bending angle θ when the sensor body 2 is deformed by bending while being elastically deformed (the calculation method using Equations (1) to (3) above) ignores the change in the thickness of the intermediate layer 21 (assumes that the intermediate layer 21 maintains its initial thickness at the time of deformation). An approximate value of the bending angle can also be calculated by this method.

On the other hand, from the viewpoint that the bending angle θ can be calculated with higher accuracy, it is preferable to calculate the bending angle θ in consideration of a change in thickness accompanying the stretching of the intermediate layer 21.

Hereinafter, a calculation method of the bending angle θ in consideration of a change in the thickness of the intermediate layer 21 will be described.

The intermediate layer 21 is a member made of, for example, an elastomer composition or the like. Therefore, in the following calculation method, the bending angle is calculated on the assumption that the volume of the intermediate layer 21 is constant even when the intermediate layer 21 is stretched and that there is no stretching directionality (in other words, when the intermediate layer 21 is elongated in the longitudinal direction, the ratio of contraction occurring in each of the width direction and the thickness direction is the same).

Assuming that the initial length of the intermediate layer 21 (which is the same as the respective lengths of the first reference line segment S1 and the second reference line segment S2 in the unelongated state) is L₀, the length of the intermediate layer 21 after the composite deformation (the average value of the length L_{3(S1)} of the first reference line segment S1 after the deformation and the length L_{3(S2)} of the second reference line segment S2 after the deformation) is L₁, the initial thickness of the intermediate layer 21 is t₀, and the thickness of the intermediate layer 21 after the deformation is t₁, the thickness of the intermediate layer 21 after the deformation is expressed by Equation (4) below:

$$\begin{matrix}[ Mathematical Expression 1 ] & \\ {t}_{1} = {t}_{0} \sqrt{\frac{{L}_{0}}{{L}_{1}}} & ( 4 )\end{matrix}$$

Thus, when the change in the thickness of the intermediate layer 21 is considered, the bending angle θ (rad) can be calculated by Equation (5) below:

$$\begin{matrix}[ Mathematical Expression 2 ] & \\ θ ( rad ) = \frac{{L}_{3 ( S 1 )} - {L}_{3 ( S 2 )}}{{t}_{0}} \sqrt{\frac{{L}_{1}}{{L}_{0}}} & ( 5 )\end{matrix}$$

As thus described, by calculating the bending angle in consideration of the change in the thickness of the intermediate layer 21, the bending angle at the time of deformation of the sensor body 2 (intermediate layer 21) can be measured more accurately. Therefore, adopting this method makes it possible to measure the bending angle of the measuring object with higher accuracy.

Note that the calculation method employed here calculates a change in the thickness of the intermediate layer at the time of deformation on the assumption that the volume of the intermediate layer 21 is constant even though the stretching thereof and that there is no stretching directionality. On the other hand, the preconditions for calculating the change in the thickness of the intermediate layer are not limited to the above conditions but may be set in accordance with the characteristics of the intermediate layer 21.

In the sensor body 2 of the present embodiment, as described above, capacitive sensor elements 22 (first sensor element 22A and second sensor element 22B) are laminated on both surfaces of the intermediate layer 21, and based on the capacitance of the detection portion 16 provided in the sensor element 22, the length of the first reference line segment S1 and the length of the second reference line segment S2 of the intermediate layer 21 are calculated.

Therefore, in the calculation method of the bending angle in the present embodiment described above, the length calculated from the measured value of the capacitance of the detection portion 16 is determined to be the length equivalent to the lengths of the first and second reference line segments S1, S2, without considering the distance in the thickness direction between the detection portion 16 of the sensor element 22 and the first and second reference line segments S1, S2 set on both surfaces of the intermediate layer 21. Even when this method is adopted, the bending angle can be measured.

On the other hand, when the sensor elements 22 are laminated on both surfaces of the intermediate layer 21, since the detection portion 16 of the sensor element 22 is separated from both surfaces of the intermediate layer 21, the amount of deformation of the detection portion at the time of bending deformation of the sensor body 2 (intermediate layer 21) is large as compared to the amounts of deformation of the first reference line segment S1 and the second reference line segment S2. This is because the detection portion 16 is separated from the surface of the intermediate layer 21 by the total thickness of the thickness of the bottom protective layer 15B and the thickness of the adhesive layer (limited to the case of having the adhesive layer) for fixing the sensor element 22 to the intermediate layer 21.

Thus, at the time of calculating the amounts of deformation of the first reference line segment S1 and the second reference line segment S2 based on the capacitance of the detection portion as in the present embodiment, it is preferable to perform correction in consideration of the total thickness. This enables the calculation of the bending angle θ with high accuracy. Specifically, the initial thickness and the thickness after the deformation, which are used for calculating the bending angle θ, may be set based on the total thickness rather than the thickness of the intermediate layer alone.

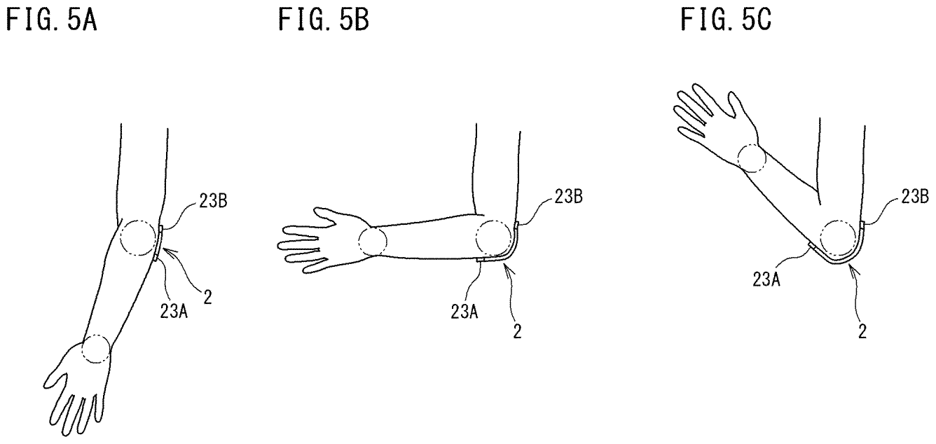

The sensor body 2 according to the present embodiment can be used, for example, for measuring the bending angle of the elbow of the human body. FIGS. 5A to 5C are views for explaining an example of the usage of the sensor body according to the present embodiment.

As shown in FIG. 5A, the sensor body 2 is placed on the outer part of the elbow. In the sensor body 2, an adhesive layer is provided on the lower surface side of the attachment members 23A, 23B and fixed to the elbow joint part by the adhesive layer.

At this time, the sensor body 2 is placed such that the first reference plane RS1 (the upper surface of the attachment member 23A) is substantially parallel to the humeral axis and that the second reference plane RS2 (the upper surface of the attachment member 23B) is substantially parallel to the ulnar axis.

In this state, the elbow is bent from the state shown in FIG. 5A to the states shown in FIGS. 5B and 5C. Then, the sensor body 2 (intermediate layer 21) is deformed in accordance with the bending state of the elbow. As a result, the bending angle of the sensor body 2 can be measured, and since the first reference plane RS1 and the humeral axis are substantially parallel and the second reference plane RS2 and the ulnar axis are substantially parallel as described above, the bending angle of the elbow joint can be obtained based on the bending angle of the sensor body 2.

The elbow joint described above is a joint with a one-axis degree of freedom.

On the other hand, the sensor body 2 can be suitably used for measurement of a bending angle of a joint having two or more axes of degrees of freedom, as well as a joint having a one-axis degree of freedom.

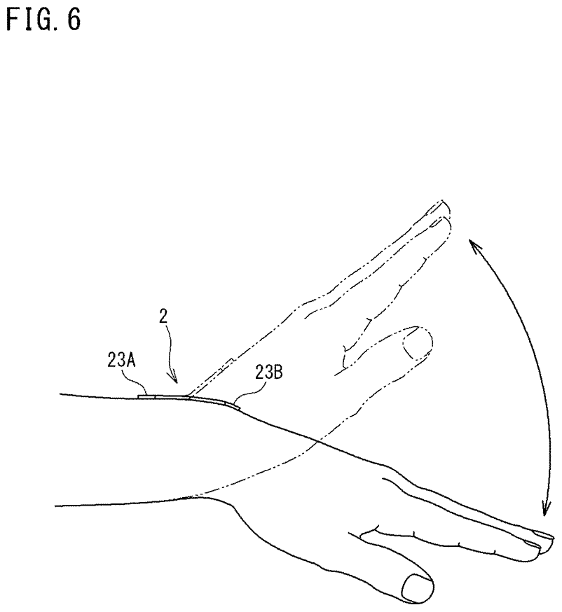

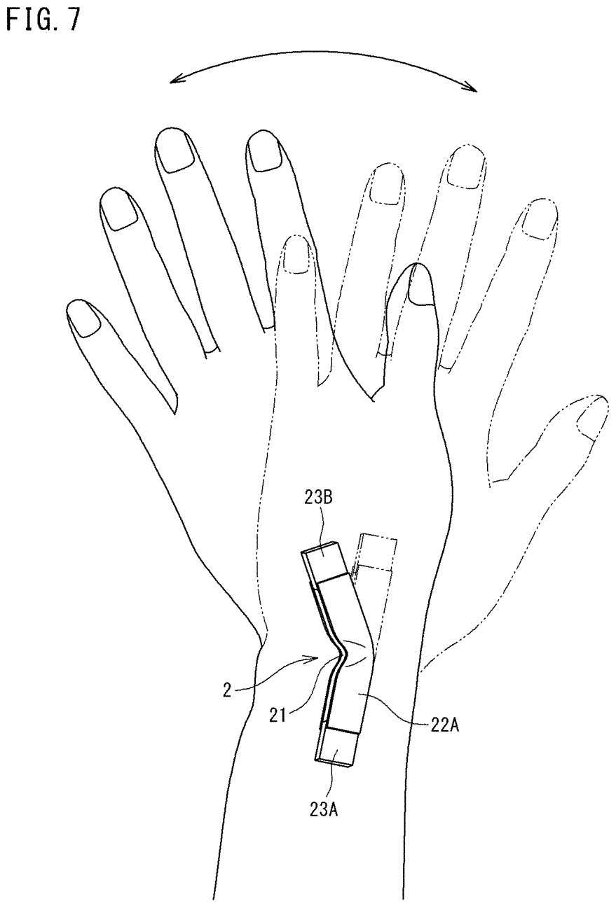

More specifically, the sensor body 2 according to the present embodiment can also be used for measuring the bending angle of the wrist of the human body. FIGS. 6 and 7 are diagrams for explaining another example of the usage of the sensor body according to the present embodiment.

As shown in FIG. 6, the sensor body 2 is placed on the back of the hand. The sensor body 2 is provided with an adhesive layer on the lower surface side of the attachment members 23A, 23B, and is fixed to the back of the hand by the adhesive layer.

At this time, the sensor body 2 is placed, for example, such that the first reference plane RS1 (the upper surface of the attachment member 23A) is substantially parallel to the ulnar axis and that the second reference plane RS2 (the upper surface of the attachment member 23B) is substantially parallel to the third metacarpal axis.

In this state, the bending (palm flexion and dorsiflexion) of the wrist is performed such that the palms come into a state shown in FIG. 6. Then, the sensor body 2 (intermediate layer) is deformed in accordance with the bending state of the wrist. As a result, the bending angle of the sensor body 2 can be measured, and since the first reference plane RS1 and the ulnar axis are substantially parallel and the second reference plane RS2 and the third metacarpal axis are substantially parallel as described above, it is possible to obtain the bending angles when the palm flexion and dorsiflexion of the wrist are performed based on the bending angle of the sensor body 2.

With the wrist being a joint having two or more axes of degrees of freedom, the wrist can also be bent (radial flexion and ulnar flexion) as shown in FIG. 7. Therefore, when the radial flexion and ulnar flexion of the wrist is performed with the sensor body 2 stuck to the back of the hand as shown in FIG. 6, the intermediate layer 21 of the sensor body 2 may be deformed to buckle as shown in FIG. 7.

As shown in FIG. 7, even when the intermediate layer 21 buckles, the sensor body 2 does not affect the measured values at the time of palm flexion or dorsiflexion of the wrist. This is because the sensor body 2 calculates the bending angle based on the difference between the lengths of the first reference line segment S1 and the second reference line segment S2 on both surfaces of the intermediate layer 21.

Therefore, the sensor body 2 according to the present embodiment is also suitable for obtaining the bending angle of the joint having two or more axes of degrees of freedom, such as the wrist joint.

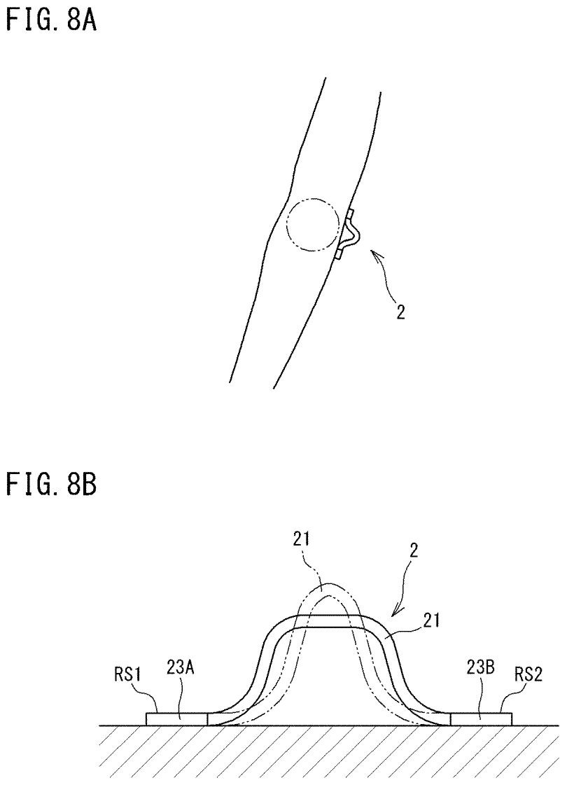

In the sensor body 2 according to the present embodiment, as shown in FIGS. 5A to 5C, when the sensor body 2 is placed on the outer part of the elbow to measure the bending angle of the elbow joint, the placement position of the sensor body 2 may be shifted in use, and the intermediate layer 21 is slackened as shown in FIG. 8A.

The shift of the placement position of the sensor body 2 may be caused by the following.

For example, the adhesive force of the adhesive layer may be insufficient.

Also, although the sensor body 2 can be placed via a supporter or a garment, when the sensor body 2 is placed in this manner, the supporter or the like may be loosened, and as a result, the intermediate layer 21 may be slackened.

However, in the measurement using the sensor body 2, the bending angle of the elbow joint can be accurately measured even when the intermediate layer 21 is loosened. The reason for this will be described below with reference to FIG. 8B.

FIG. 8B is a view for explaining the characteristics of the sensor body according to the embodiment of the present invention.

In the example shown in FIG. 8B, in the sensor body 2, the angle θ formed by the first reference plane RS1 (the upper surface of the attachment member 23A) and the second reference plane RS2 (the upper surface of the attachment member 23B) is constant regardless of the deformed state of the intermediate layer 21, and in FIG. 8B, the first reference plane RS1 and the second reference plane RS2 are in the same plane and θ=0.

In the sensor body 2 deformed as shown in FIG. 8B, the relationship between the length L_{4(S1)} of the first reference line segment S1 and the length L_{4(S2)} of the second reference line segment S2 of the intermediate layer 21 can be expressed by Equations (6) to (8) below, where t is the thickness of the intermediate layer 21 (here, the thickness of the intermediate layer 21 is assumed to be constant).

$$\begin{matrix} [ Mathematical Expression 3 ] & \\ \begin{matrix}{L}_{4 ( S 1 )} = {\int }_{0}^{{L}_{4 ( S 2 ) t}} \frac{{ρ}_{LS 2} + t}{{ρ}_{LS 1}} d L \\ = \frac{{ρ}_{LS 2} + t}{{ρ}_{LS 2}} {L}_{4 ( S 2 )} + const \\ = {L}_{4 ( S 2 )} + θ t\end{matrix} & \begin{matrix}\begin{matrix}( 6 ) \\ ( 7 )\end{matrix} \\ ( 8 )\end{matrix}\end{matrix}$$

(in Equations (6) and (7), ρ_{LS2} is a radius of curvature at the time of deformation of the second reference line segment S2).

In the example shown in FIG. 8B, since θ=0, the length L_{4(S1)} of the first reference line segment S1 of the intermediate layer 21 and the length L_{4(S2)} of the second reference line segment S2 are:

L_{4(S1)}=L_{4(S2)}.

As thus described, regardless of the deformed state of the intermediate layer 21, when the angle θ formed by the first reference plane RS1 and the second reference plane RS2 is 0 (rad), θt in Equation (8) becomes 0, and the length L_{4(S1)} of the first reference line segment S1 and the length L_{4(S2)} of the second reference line segment S2 become the same. Therefore, even when the intermediate layer 21 is slackened, the angle θ between the first reference plane RS1 and the second reference plane RS2 can be measured as the bending angle of the sensor body 2 from the difference between L_{4(S1)} and L_{4(S2)} and the thickness t of the intermediate layer 21.

FIG. 8B shows the case where the angle θ formed by the first reference plane RS1 and the second reference plane RS2 is 0 (rad), but an arbitrary bending angle θ(rad) formed by the first reference plane RS1 and the second reference plane RS2 can be obtained based on Equation (9) below obtained by transforming Equation (8):

$$\begin{matrix}[ Mathematical Expression 4 ] & \\ θ ( rad ) = \frac{{L}_{4 ( S 1 )} - {L}_{4 ( S 2 )}}{t} & ( 9 )\end{matrix}$$

For performing the above-described measurement using the sensor body 2, the sensor device 1 includes the analyzer 3.

The analyzer 3 includes a detection circuit 3a for measuring the capacitance of the detection portion 16 of each of the first sensor element 22A and a second sensor element 22B provided in the sensor body 2, and an operational portion 3b for calculating the length of each of the first reference line segment S1 and the second reference line segment S2 based on the obtained capacitance and calculating the amount of elongation and the bending angle of the sensor body 2.

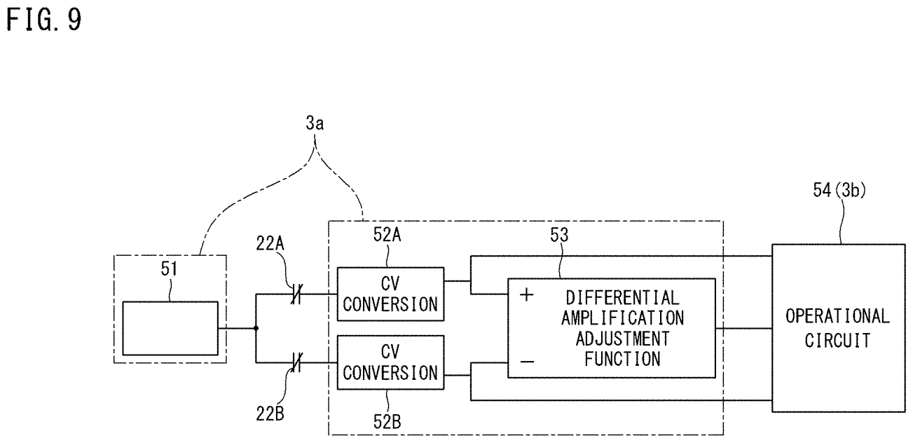

FIG. 9 is a view for explaining an example of the detection circuit provided in the analyzer of the sensor device according to the embodiment of the present invention.

The detection circuit 3a includes an oscillation circuit 51 for generating carriers, capacitance-to-voltage (CV) conversion circuits 52A, 52B connected to the sensor elements 22A, 22B, respectively, and a circuit 53 having a differential amplification adjustment function.

The detection circuit 3a converts capacitances C detected in the respective detection portions of the first sensor element 22A and the second sensor element 22B into voltage signals V, amplifies the difference between electric signals obtained from the first sensor element 22A and the second sensor element 22B, and outputs the amplified electric signal.

The signal output from the detection circuit 3a is input to an operational circuit 54 (operational portion 3b) and used for calculating the amount of elongation and the bending angle of the sensor body 2.

The configuration of the detection circuit for measuring the capacitance in the analyzer 3 is not limited to such a configuration.

The indicator 4 includes a monitor 4a and a storage portion 4b.

In the indicator 4, the monitor 4a indicates measurement results such as the amount of elongation and the bending angle of the measuring object. The storage portion 4c stores the measurement results and data used for calculating the amount of elongation and the bending angle.

Note that a terminal device such as a personal computer, a smartphone, or a tablet may be used as the operational portion 3b and the indicator 4.

(Second Embodiment)

The sensor body according to the present embodiment of the present invention is not limited to the sensor body 2 according to the first embodiment.

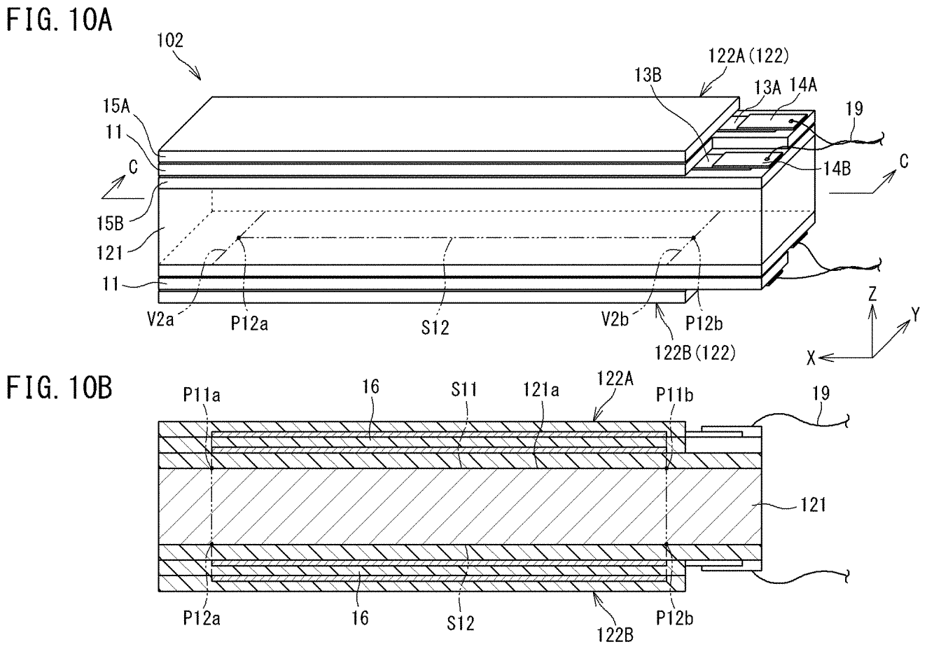

FIG. 10A is a perspective view showing another example of the sensor body according to the embodiment of the present invention, and FIG. 10B is a cross-sectional view taken along line C-C of FIG. 10A.

A sensor body 102 according to the present embodiment is different from the sensor body of the sensor device according to the first embodiment in that attachment members are not provided at both ends of the intermediate layer.

As shown in FIGS. 10A and 10B, the sensor body 102 is provided with a plate-like intermediate layer 121 and two sensor elements 122 (first sensor element 122A and second sensor element 122B) laminated on both surfaces (the upper surface and lower surface) of the intermediate layer 121.

Here, the two sensor elements 122 (first sensor element 122A and second sensor element 122B) provided in the sensor body 102 are the same as the first sensor element 22A and the second sensor element 22B provided in the sensor body 2 of the first embodiment.

In the description of the present embodiment as well, as shown in FIG. 10A, the longitudinal direction of the intermediate layer 121 is referred to as the X-direction (the horizontal direction in FIG. 10A), the thickness direction of the intermediate layer 121 is referred to as the Z-direction, and the direction perpendicular to the X-direction and the Z-direction is referred to as the Y-direction.

The intermediate layer 121 is a similar member to the intermediate layer 21.

The first sensor element 122A and the second sensor element 122B are laminated on both surfaces of the intermediate layer 121 via a flexible adhesive layer (not shown). Note that the adhesive layer is not essential in the present embodiment as well.

In the sensor body 102, a center region of the upper surface of the intermediate layer 121 is set with a first reference line segment S11. The first reference line segment S11 is set so as to pass through a position dividing the upper surface of the intermediate layer 121 into two sections in the Y-direction. On the lower surface of the intermediate layer 121, a second reference line segment S12 is set to pass through a position dividing the lower surface into two sections in the Y-direction. The second reference line segment S12 is set at a position overlapping with the first reference line segment S11 in the thickness direction (Z-direction), and the second reference line segment S12 is parallel to the first reference line segment S11.

The first sensor element 122A laminated on the upper surface of the intermediate layer 121 is laminated on the intermediate layer 121 such that both end edges in the X-direction of the detection portion 16 of the first sensor element 122A overlap with two imaginary lines, passing through the end points P11a, P11b of the first reference line segment S11 and extending in the Y-direction, in the thickness direction (Z-direction) of the sensor body 102.

The second sensor element 122B laminated on the lower surface of the intermediate layer 121 is laminated on the intermediate layer 121 such that both end edges of the detection portion 16 of the second sensor element 122B in the X-direction overlap with two imaginary lines V2a, V2b, passing through end points P12a, P12b of the second reference line segment S12 and extending in the Y-direction, in the thickness direction (Z-direction) of the sensor body 102.

Therefore, in the sensor body 102, the first sensor element 122A is provided such that the first reference line segment S11 set on the upper surface of the intermediate layer 121 overlaps with the detection portion 16 in the thickness direction, and the second sensor element 122B is provided such that the second reference line segment S12 set on the lower surface of the intermediate layer 121 overlaps with the detection portion 16 in the thickness direction.

As in the case of the sensor body 2, the sensor body 102 can calculate the amount of elongation of the sensor body 102 (intermediate layer 121) due to elastic deformation as an average value between the amount of change in the length of the first reference line segment S11 and the amount of change in the length of the second reference line segment S12.

Further, in the sensor body 102, an arbitrary region, which is in the same plane as an upper surface 121a of the intermediate layer 121, includes the end point P11a of the first reference line segment S11, and is outside the end point P11a (opposite to the first reference line segment S11 side), is taken as a first reference plane (not shown). Also, an arbitrary region, which is in the same plane as the upper surface 121a of the intermediate layer 121, includes the end point P11b of the first reference line segment S11, and is outside the end point P11b, is taken as a second reference plane (not shown) In this case, a predetermined part of the upper surface of the intermediate layer 121 corresponds to the first reference plane, and a predetermined another part of the upper surface of the intermediate layer 121 corresponds to the second reference plane.

The sensor body 102 calculates the bending angle of the sensor body 102 as the angle formed by the first reference plane and the second reference plane.

The sensor body 102 can grasp a change in the length of the central part of the intermediate layer 121, which occurs at the time of deformation of the intermediate layer 121, as an amount of elongation and can grasp the magnitude of an angle formed by the first reference plane and the second reference plane as a bending angle.

Therefore, as in the case of the sensor body 2, the sensor body 102 can measure the amount of elongation and the bending angle of the measuring object by being placed on a predetermined position of the measuring object and measuring the amount of elongation and the bending angle which occur at the time of deformation of the sensor body 102 (intermediate layer 121).

Here, the method for measuring the amount of elongation and the bending angle of the measuring object by using the sensor body 102 of the present embodiment is the same as that of the first embodiment.

The sensor body 102 includes an adhesive layer (not shown) for sticking the sensor body 102 to a measuring object on the lower surface side. For example, the adhesive layer may be provided only at positions overlapping with the first reference plane and the second reference plane in the Z-direction.

(Third Embodiment)

In the sensor body according to the embodiment of the present invention, the sensor element provided in the sensor body is not limited to the sensor element 22 but may be a sensor element including a second dielectric layer and a third electrode layer in addition to the dielectric layer (first dielectric layer), and the first electrode layer and the second electrode layer which are formed on both surfaces of the dielectric layer.

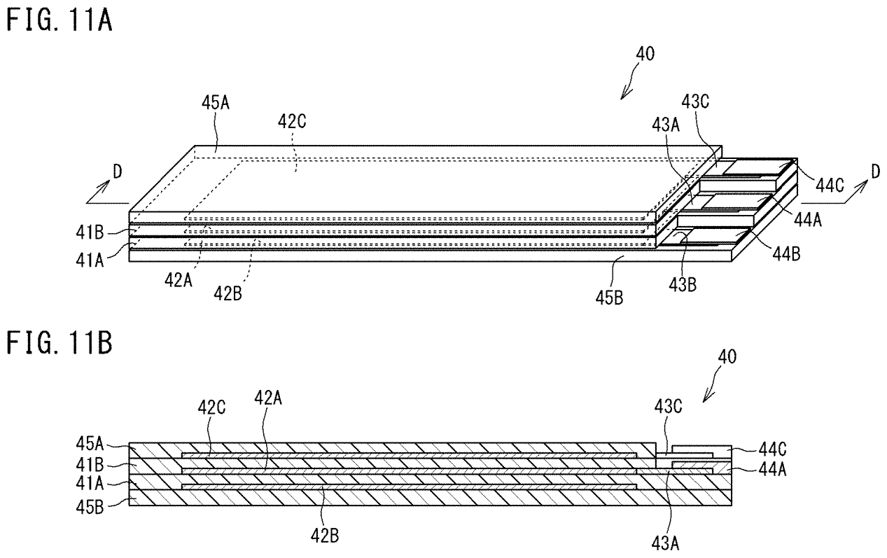

FIG. 11A is a perspective view showing another example of the sensor element provided in the sensor body according to the embodiment of the present invention, and FIG. 11B is a cross-sectional view taken along line D-D in FIG. 11A.

The capacitive sensor element 40 shown in FIGS. 11A and 11B includes a sheet-like first dielectric layer 41A made of an elastomer composition, a first electrode layer 42A formed on the top surface of the first dielectric layer 41A, a second electrode layer 42B formed on the bottom surface of the first dielectric layer 41A, a second dielectric layer 41B laminated on the top of the first dielectric layer 41A so as to cover the first electrode layer 42A, and a third electrode layer 42C formed on the top surface of the second dielectric layer 41B.

The sensor element 40 includes a first conducting wire 43A coupled to the first electrode layer 42A, a second conducting wire 43B coupled to the second electrode layer 42B, a third conducting wire 43C coupled to the third electrode layer 42C, a first connecting portion 44A attached to the end of the first conducting wire 43A on the opposite side to the first electrode layer 42A, a second connecting portion 44B attached to the end of the second conducting wire 43B on the opposite side to the second electrode layer 42B, and a third connecting portion 44C attached to the end of the third conducting wire 43C on the opposite side to the third electrode layer 42C.

In the sensor element 40, a bottom protective layer 45B and a top protective layer 45A are provided on the bottom side of the first dielectric layer 41A and the top side of the second dielectric layer 41B, respectively.

The first electrode layer 42A to the third electrode layer 42C have the same shape in a plan view. The first electrode layer 42A and the second electrode layer 42B as a whole face each other across the first dielectric layer 41A, and the first electrode layer 42A and the third electrode layer 42C face each other as a whole across the second dielectric layer 41B.

In the sensor element 40, a part where the first electrode layer 42A and the second electrode layer 42B face each other and a part where the first electrode layer 42A and the third electrode layer 42C face each other are a detection portion, and the sum of the capacitance of the part where the first electrode layer 42A and the second electrode layer 42B face each other and the capacitance of the part where the first electrode layer 42A and the third electrode layer 42C face each other is the capacitance of the detection portion.

The sensor body provided with such a sensor element 40 is suitable for more accurately measuring a change in capacitance by eliminating a decrease in measurement accuracy caused by a noise source, such as the sensor body being close to a living body as a conductor, which may occur when the sensor body is stuck to the skin surface of the living body.

(Fourth Embodiment)

The sensor body according to the present embodiment of the present invention may further include a reinforcing member in the sensor body 102 according to the second embodiment.

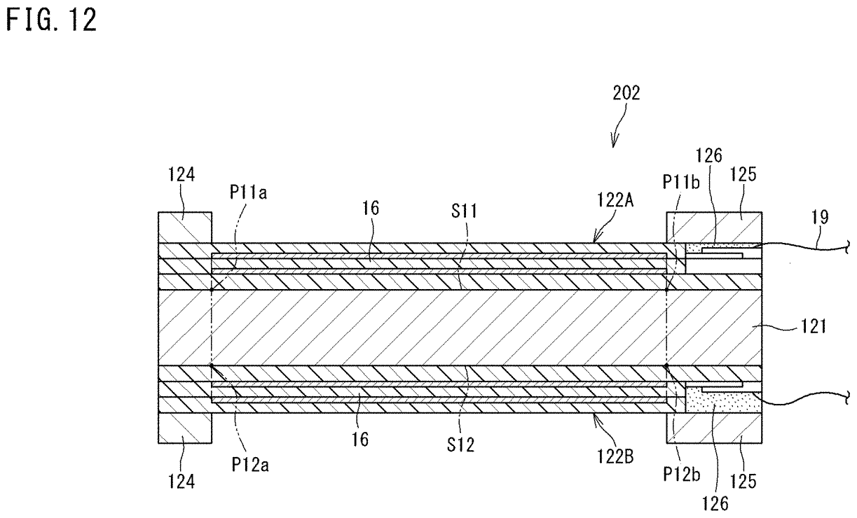

FIG. 12 is a cross-sectional view showing another example of the sensor body according to the embodiment of the present invention.

As shown in FIG. 12, a sensor body 202 according to the present embodiment has a similar configuration to that of the sensor body 102 according to the second embodiment and further includes reinforcing members 124, 125. In FIG. 12, the same members as those of the sensor body 102 according to the second embodiment are denoted by the same reference numerals.

The sensor body 202 includes a plate-like intermediate layer 121 and two sensor elements (first sensor element 122A and second sensor element 122B) laminated on both surfaces (the upper surface and lower surface) of the intermediate layer 121. The sensor body 202 further includes the reinforcing members 124, 125 at both ends in the longitudinal direction (X-direction) of the intermediate layer 121.

The reinforcing members 124, 125 have higher rigidity than the intermediate layer 121 made of polyethylene terephthalate (PET) or the like. The reinforcing members 124, 125 are preferably members that do not substantially deform under the conditions of use. Examples of the material of the reinforcing members 124, 125 includes one similar to the material of the attachment members 23A, 23B provided in the sensor body 2.

The reinforcing members 124, 125 are laminated at the respective predetermined positions on both sensor elements (first sensor element 122A and second sensor element 122B) laminated on both surfaces of the intermediate layer 121. The reinforcing members 124, 125 are fixed via adhesive layers. In FIG. 12, reference numeral 126 denotes an adhesive layer for fixing the reinforcing member 125.

The reinforcing members 124, 125 are laminated in such positions where the reinforcing members 124, 125 do not overlap with the first reference line segment S11, set on the upper surface of the intermediate layer 121, and the second reference line segment S12, set on the lower surface of the intermediate layer 121 in the thickness direction Z-direction, and where two imaginary lines passing through the end points P11a, P11b of the first reference line segment S11 and extending in the Y-direction (or two imaginary lines V2a, V2b passing through the end points P12a, P12b of the second reference line segment S12 and extending in the Y-direction (cf. FIG. 10A) overlap with the edges of the reinforcing members 124, 125 on the reference line segment sides in the thickness direction.

At the time of deformation of the sensor body 202 including the reinforcing members 124, 125, the sensor body 202 can prevent the first reference plane and/or the second reference plane, set in the parts of the region of the intermediate layer 121, from being deformed in such a manner as to be bent, elongated, or contracted.

Therefore, according to the sensor body 202 of the present embodiment, it is possible to grasp the deformed state, such as the amount of elongation and the bending angle, of the sensor body 202 with higher accuracy.

Therefore, the sensor body 202 can measure the amount of elongation and the bending angle of the measuring object by being placed on a predetermined position of the measuring object and measuring the amount of elongation and the bending angle which occur at the time of deformation of the sensor body 102 (intermediate layer 121).

The sensor body 202 according to the present embodiment is provided with the reinforcing members 124, 125 on the upper surface side and the lower surface side of the intermediate layer 121, respectively, but when the sensor body 202 according to the embodiment of the present invention includes the reinforcing member, the reinforcing member may be provided only on one surface side of the intermediate layer 121.

Next, a description will be given of the constituent members of the sensor device according to the embodiment of the present invention described above.

[Sensor Body]

<Intermediate Layer>

The intermediate layer is a member that can be freely deformed in accordance with an external force when the external force is applied, while maintaining its shape in a natural state. The material of the intermediate layer is not particularly limited but may be a member such as an elastomer or cloth member.

When the intermediate layer is made of an elastomer, the intermediate layer is a sheet-like product formed using an elastomer composition.

The elastomer composition includes, for example, an elastomer and, when necessary, other optional components.

Examples of the elastomer include natural rubber, isoprene rubber, nitrile rubber (NBR), ethylene propylene rubber (EPDM), styrene-butadiene rubber (SBR), butadiene rubber (BR), chloroprene rubber (CR), silicone elastomer, fluorine rubber, acrylic rubber, hydrogenated nitrile rubber, and urethane elastomer. These may be used alone or in a combination of two or more.

The elastomer composition may contain additives such as a plasticizer, an antioxidant, an age resistor, and a coloring agent as necessary in addition to the elastomer.

The material of the intermediate layer is preferably the same as the material of the protective layer constituting the sensor element from the viewpoint of facilitating correction in consideration of the thicknesses of the intermediate layer and the sensor element.

When the intermediate layer is made of cloth, the intermediate layer is made of cloth material having sufficient stretchability. At this time, the cloth material may be a cloth material having stretching isotropy or a cloth material having stretching anisotropy.

The intermediate layer may be a plate-like body having two parallel surfaces facing each other on which the first sensor element and the second sensor element are laminated.

The shape of the intermediate layer in a plan view is not particularly limited but is preferably a rectangular shape. In this case, the attachment members can be easily fixed to both ends. The size of the shape of the intermediate layer in the plan view is not particularly limited but may be nearly the same as the size of the sensor element laminated on the intermediate layer or may be larger than the size of the sensor element.

The size of the intermediate layer is not particularly limited but may be appropriately selected in accordance with the type of the measuring object and the measurement place.

The thickness of the intermediate layer is preferably from 1 to 10 mm. When the thickness of the intermediate layer is less than 1 mm, the difference between the amount of deformation of the first reference line segment S1 and the amount of deformation of the second reference line segment S2 at the time of bending deformation is small, which may result in inferior measurement accuracy. On the other hand, when the thickness of the intermediate layer exceeds 10 mm, the deformation of the intermediate layer itself may be inhibited.

In the intermediate layer, the sensor element may be laminated (integrated) via an adhesive or may be directly laminated. In the latter case, for example, the material of the intermediate layer may be cast between the sensor elements in a mold, and the intermediate layer and the sensor element may be integrated at the same time as the molding of the intermediate layer. Further, the intermediate layer and the sensor element may be stuck to each other by utilizing the stickiness of each of the materials thereof.

<Sensor Element>

The capacitive sensor element will be described below.

«Dielectric Layer»

The capacitive sensor element includes a dielectric layer made of elastomer.

The dielectric layer is a sheet-like product formed using an elastomer composition and can be reversibly deformed such that the areas of the top and bottom surfaces of the dielectric layer change. Therefore, the dielectric layer can be deformed in the surface direction. In the embodiment of the present invention, the top and bottom surfaces of the dielectric layer mean the top surface and the bottom surface of the dielectric layer.

The elastomer composition includes, for example, an elastomer and, when necessary, other optional components.

Examples of the elastomer include natural rubber, isoprene rubber, nitrile rubber (NBR), ethylene propylene rubber (EPDM), styrene-butadiene rubber (SBR), butadiene rubber (BR), chloroprene rubber (CR), silicone elastomer, fluorine rubber, acrylic rubber, hydrogenated nitrile rubber, and urethane elastomer. These may be used alone or in a combination of two or more.

Among these, urethane elastomer and silicone elastomer rear preferred in view of small permanent strain (or permanent amount of elongation). In terms of excellent adhesion to carbon nanotubes, urethane elastomer is preferred.

The elastomer composition may contain additives such as a plasticizer, an antioxidant, an age resistor, and a coloring agent, a dielectric filler, and the like as necessary in addition to the elastomer.

The average thickness of the dielectric layer is preferably from 10 to 1000 μm, from the viewpoint of increasing the capacitance to improve the detection sensitivity. The average thickness is more preferably from 30 to 200 μm.

The dielectric layer is preferably deformable so as to increase the areas of the top and bottom surfaces by 30% or more from the non-elongated state at the time of deformation of the dielectric layer. In this case, the dielectric layer is suitable for deformation following the deformation of the living body surface, for example.

Being deformable so as to increase the areas by 30% or more means that not breaking even when a load is applied to increase the area by 30% and returning to the original state when the load is released (i.e., being in the elastic deformation range).

The dielectric layer is more preferably deformable so as to increase the areas of the top and bottom surfaces by 50% or more, even more preferably deformable so as to increase the areas by 100% or more, and particularly preferably deformable so as to increase the areas by 200% or more.

The range of the area in which the dielectric layer is deformable can be controlled by the design (material, shape, etc.) of the dielectric layer.

«Electrode Layer»

The capacitive sensor element includes the electrode layer made of an electroconductive composition containing a conductive material.

Here, each of the electrode layers may be made of an electroconductive composition having the same composition or may be made of an electroconductive composition having a different composition.

Examples of the conductive material include carbon nanotubes, graphene, carbon nanohorns, carbon fibers, electroconductive carbon black, graphite, metallic nanowires, metallic nanoparticles, and electroconductive polymers. These may be used alone or in a combination of two or more.

The conductive material is preferably carbon nanotubes. This is because the conductive material is suitable for forming an electrode layer which is deformed following the deformation of the dielectric layer.

Known carbon nanotubes can be used as the carbon nanotubes. The carbon nanotube may be single-walled carbon nanotubes (SWNTs), double-walled carbon nanotubes (DWNTs), or multi-walled carbon nanotubes (MWNT) having three or more layers (in the present specification, both of double-walled carbon nanotubes and the multi-walled carbon nanotubes are simply referred to as multi-walled carbon nanotubes). Further, two or more types of carbon nanotubes having different numbers of layers may be used in a combination.

The shape (average length, fiber diameter, aspect ratio) of each carbon nanotube is not particularly limited. The shape of the carbon nanotube may be appropriately selected by comprehensively judging the electric conductivity and durability required for the capacitive sensor element, as well as the processing and cost for forming the electrode layer.

In addition to the conductive material, the electroconductive composition may contain, for example, a binder component functioning as a connecting material for the conductive material, various additives, and the like.

Examples of the additive include a dispersant for a conductive material, a crosslinking agent for a binder component, a vulcanization accelerator, a vulcanization assistant, an age resistor, a plasticizer, a softener, and a coloring agent.

«Protective Layer»

In the capacitive sensor element, the protective layer (top protective layer and bottom protective layer) is preferably laminated. By providing the protective layer, the electrode layer and the like can be electrically insulated from the outside. By providing the protective layer, the strength and durability of the capacitive sensor element can be enhanced.

Examples of the material of the protective layer include a similar elastomer composition to the material of the dielectric layer.

«Others»

As shown in FIG. 2 and the like, each conducting wire connected to each electrode layer is normally formed in the capacitive sensor element.

Each conducting wire may be any conducting wire which does not inhibit the deformation of the dielectric layer and maintains electric conductivity even when the dielectric layer is deformed, and examples thereof include a conducting wire made of a similar electroconductive composition to that of the electrode layer.

Further, as shown in FIG. 2 and the like, a connecting portion for connecting to an external conducting wire is normally formed at the opposite end of each conducting wire to the electrode layer. Examples of each connecting portion include one formed using copper foil or the like.

Such a capacitive sensor element can be manufactured by a known method such as a similar method to a method for producing a sensor sheet described in Japanese Unexamined Patent Publication No. 2016-90487.

<Adhesive Layer>

The sensor body may include, on the outermost layer on the lower surface side, an adhesive layer for sticking the sensor body to the measuring object such as the living body surface.

The adhesive layer is not particularly limited, but examples thereof include a layer made of an acrylic pressure-sensitive adhesive, a urethane pressure-sensitive adhesive, a rubber pressure-sensitive adhesive, a silicone pressure-sensitive adhesive, and the like.

Here, each pressure-sensitive adhesive may be of a solvent type, an emulsion type, or a hot-melt type.

The adhesive layer may be provided on the entire lower surface side of the sensor body or may be provided only on a part of the lower surface side of the sensor body.

Here, when the sensor element is the capacitive sensor element described above, the adhesive layer is preferably provided as shown in (1) or (2) below. That is:

(1) the adhesive layer is provided only at a position not overlapping with the detection portion of the capacitive sensor element in the thickness direction of the sensor body; and

(2) the adhesive layer provided at a position overlapping with the detection portion of the capacitive sensor element in the thickness direction of the sensor body is a flexible adhesive layer.

[Analyzer]

The analyzer is connected to the sensor body. The analyzer measures a change in the length corresponding to the length of the first reference line segment and a change in the length corresponding to the length of the second reference line segment based on the capacitance of the detection portion of each sensor element.

The method for measuring the capacitance is not particularly limited, but the capacitance can be measured using various known methods in addition to the method using the detection circuit 3a described above.

(Other Embodiments)

The embodiment of the present invention is not limited to the embodiment described above but may be appropriately modified within the scope of the claims.

The sensor element provided in the sensor body according to the embodiment of the present invention is not limited to the capacitive sensor element described above.

The sensor element may be any element capable of measuring a change in the respective lengths of the first reference line segment and the second reference line segment set in the intermediate layer and not substantially inhibiting the deformation of the sensor body itself or the deformation of the measuring object.

The first sensor element and the second sensor element, which are laminated on both surfaces of the intermediate layer, are preferably the same as in the first embodiment. In this case, for example, even when a temperature change occurs during measurement, the measurement can be performed with high accuracy.

In the embodiment described above, the method of directly sticking the sensor body 2 to the skin surface of the human body has been mainly described, but in a case where the bending angle of the joint of the human body, or the like, is measured using the sensor body 2 according to the embodiment of the present invention, the sensor body 2 may be placed on the human body via clothing, a supporter, or the like.

### EXAMPLE

The present invention will be described more specifically with reference to the following examples, but the present invention is not limited to the following examples.

### Example 1

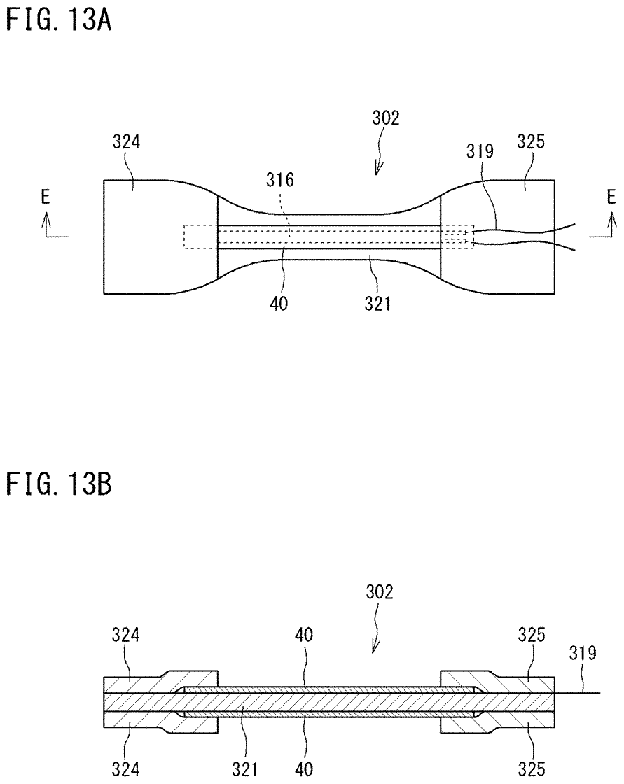

In the present example, a sensor body shown in FIGS. 13A and 13B was produced, and its characteristics were evaluated. FIG. 13A is a plan view schematically showing a sensor body produced in the present example, and FIG. 13B is a cross-sectional view taken along line E-E in FIG. 13A.

«Manufacture of Sensor Elements»

Here, two sensor elements 40 shown in FIGS. 11A and 11B were produced by the following method.

(1) Production of Dielectric Layers (First Dielectric Layer 41A and Second Dielectric Layer 41B)

100 parts by weight of a polyol (Pandex GCB-41, manufactured by DIC Corporation) was added to 40 parts by weight of a plasticizer (dioctyl sulfonate) and 17.62 parts by weight of an isocyanate (Pandex GCA-11, manufactured by DIC Corporation), which was then agitated and mixed with an agitator for 90 seconds to prepare a raw material composition for a dielectric layer.

Next, the raw material composition was heated in a heating device (crosslinking furnace) while being conveyed in a state where the raw material composition was sandwiched between two protective films. Here, crosslinking was performed under the conditions of an internal temperature in a furnace of 70° C. and an internal time in the furnace of 30 minutes to obtain a roll-wound sheet having a predetermined thickness with a protective film. Thereafter, crosslinking was performed after 12 hours in a furnace adjusted to 70° C. to produce a sheet having a thickness of 100 μm made of a polyether-based urethane elastomer. The obtained sheet was cut to produce two sheets having a thickness of 75 mm×8 mm×100 μm (thickness). One corner portion of one cut sheet was cut off to a size of 8 mm×2.6 mm×100μm (thickness) to produce a first dielectric layer. One corner portion of the other one cut sheet was cut off to a size of 8 mm×5.3 mm×100 μm (thickness) to produce a second dielectric layer.

The elongation (%) at break and the relative permittivity of the produced dielectric layer were measured. The elongation (%) at break was 505%, and the relative permittivity was 5.8.

The elongation at break was measured in accordance with JIS K 6251. The tensile speed was set to 500 mm/min.

The dielectric layer was sandwiched between electrodes having diameters of 20 mm, and the capacitance was measured at a measurement frequency of 1 kHz by using an LCR high tester (3522-50, manufactured by HIOKI E.E. Corporation) to calculate the relative permittivity from the electrode area and the thickness of the measurement sample.

(2) Preparation of Electrode Layer Material

30 mg of highly oriented carbon nanotubes (4 to 12 layers, fiber diameters of 10 to 20 nm, fiber lengths of 150 to 300 μm, and carbon purities of 99.5%), manufactured by TAIYO NIPPON SANSO CORPORATION, was added to 30g of isopropyl alcohol (IPA), which was subjected to wet dispersion treatment using a jet mill (Nanojet Pal JN10-SP003, manufactured by JOKOH CO., LTD.) and diluted twice to obtain a carbon nanotube dispersion liquid having a concentration of 0.05% by weight.

(3) Production of Protective Layers (Top Protective Layer 45A and Bottom Protective Layer 45B)

By using a similar method to that for producing the dielectric layer (1) described above, a bottom protective layer of 75 mm×8 mm×100 μm (thickness) and a top protective layer of 67 mm×8 mm×100 μm (thickness) made of polyether-based urethane elastomer were produced.

(4) Production of Capacitive Sensor Element 40

The capacitive sensor element 40 shown in FIGS. 11A and 11B was produced through the following production steps (a) to (e).

(a) A mask (not shown) having an opening of a predetermined shape formed on a PET film subjected to releasing treatment was stuck to one surface (top surface) of the bottom protective layer 45B produced in the above step (3).

An opening corresponding to the second electrode layer and the second conducting wire is formed in the mask, and the size of the opening is 5 mm in width and 50 mm in length for the part corresponding to the second electrode layer, and 1.5 mm in width and 6 mm in length for the part corresponding to the second conducting wire.

Next, the carbon nanotube dispersion liquid prepared in the above step (2) was applied with an airbrush such that the amount of application per unit area (cm²) was 0.223 g. Subsequently, drying was performed at 100° C. for ten minutes to form the second electrode layer 42B and the second conducting wire 43B. Thereafter, the mask was peeled off.

(b) The first dielectric layer 41A produced in the above step (1) was laminated on the bottom protective layer 45B so as to cover the entire second electrode layer 42B and a part of the second conducting wire 43B.

Further, the carbon nanotube dispersion liquid was applied to the top of the first dielectric layer 41A by using the same method as that employed in the above step (a) and dried to form the first electrode layer 42A and the first conducting wire 43A at predetermined positions (positions where the second electrode layer 42B and the first electrode layer 42A overlap when seen in a plan view).

(c) The second dielectric layer 41B produced in the above step (1) was laminated on the first dielectric layer 41A so as to cover the entire first electrode layer 42A and a part of the first conducting wire 43A.

Further, the carbon nanotube dispersion liquid was applied to the top of the second dielectric layer 41B by using the same method as that employed in the above step (a) and dried to form the third electrode layer 42C and the third conducting wire 43C at predetermined positions (positions where the third electrode layer 42C and the first electrode layer 42A overlap when seen in a plan view).

(d) The top protective layer 45A produced in the above step (3) was laminated on the top of the second dielectric layer 41B, on which the third electrode layer 42C and the third conducting wire 43C were formed, so as to cover the entire third electrode layer 42C and a part of the third conducting wire 43C.

(e) Thereafter, copper foil was attached to each end of the first conducting wire 43A, the second conducting wire 43B, and the third conducting wire 43C to form the first connecting portion 44A, the second connecting portion 44B, and the third connecting portion 44C.

Next, a lead wire serving as an external conducting wire was fixed to each of the first connecting portion 44A, the second connecting portion 44B, and the third connecting portion 44C by soldering to complete the sensor element 40.

«Manufacturing of Sensor Body»

(1) Preparation of Intermediate Layer

An intermediate layer made of urethane elastomer having the same shape as the dumbbell-shaped No. 3 type adopted in JIS K 6251 was prepared.

(2) Preparation of Reinforcing Members

A PET resin plate having a thickness of 50 μm was cut into a shape corresponding to the shape of the end of the intermediate layer to prepare a reinforcing member.

(3) Preparation of Adhesive

As an adhesive, a double-sided tape (No. 500, manufactured by Nitto Denko Corporation) was prepared.

(4) Manufacturing of Sensor Body

The sensor body shown in FIG. 13A and FIG. 13B was produced.

First, the sensor elements 40 were stuck to both surfaces (upper and lower surfaces) of an intermediate layer 321 prepared in the above step (1). The sticking was performed by utilizing the stickiness of each of the intermediate layer and the protective layer.

Next, reinforcing members 324, 325 prepared in the above step (2) were fixed to both ends in the longitudinal direction (X-direction) of the sensor elements 40, 40 stuck to both surfaces of the intermediate layer 321 by using the double-sided tapes (not shown) prepared in the above step (3). Thus, a sensor body 302 was produced.

In the sensor body 302, reference numeral 316 denotes the detection portion of the sensor element 40, and reference numeral 319 denotes a lead wire attached to the sensor element 40.

<Assembly of Sensor Device>

The lead wires 319 of each of the two sensor elements 40, 40 provided in the sensor body 302 were connected to a CV conversion substrate (original product), and the CV conversion substrate was connected to a digital multimeter (DT4282, manufactured by HIOKI E.E. CORPORATION) configured to measure an output voltage, thereby forming a sensor device.

[Evaluation]

The above sensor device was evaluated by the following method.

<1. Bending Evaluation 1>

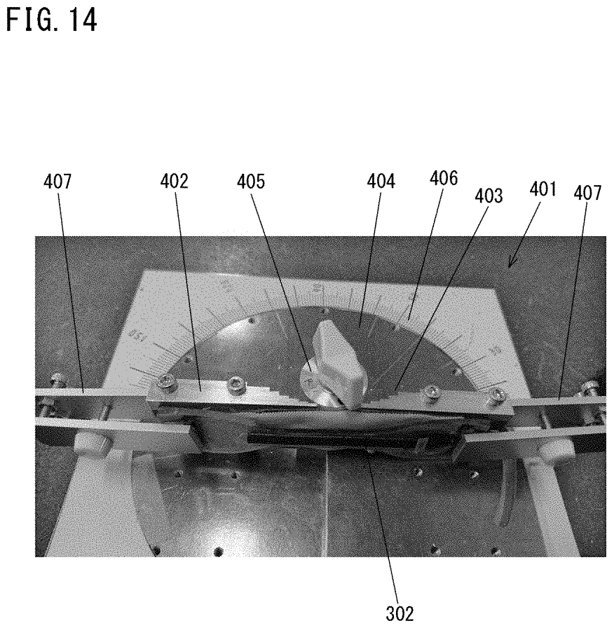

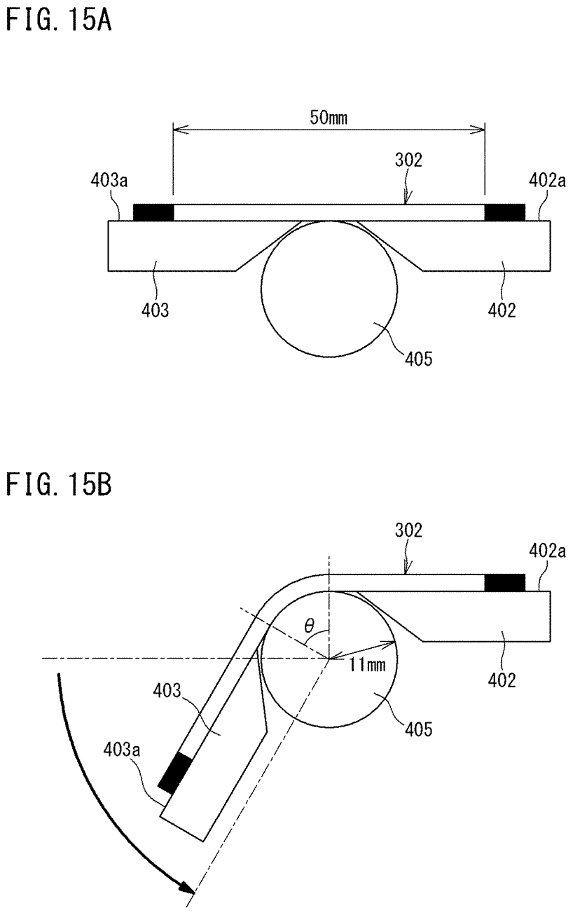

FIG. 14 is a photograph showing a measurement jig to which the sensor body has been attached. FIGS. 15A and 15B are views for explaining bending evaluation 1.

The present evaluation was performed using a measurement jig 401. As shown in FIG. 14, the measurement jig 401 includes a rotating table 404 having two levers (first lever 402 and second lever 403), and an angle scale 406 provided along the outer edge of the rotating table 404. The second lever 403 is configured to be rotatable with respect to the first lever 402. A cylindrical member 405 is provided at the center part of the rotating table 404, and the bending radius at the time of measurement can be adjusted using the size of the cylindrical member 405. In the present evaluation, the radius of the cylindrical member 405 was set to 11 mm.

In the present evaluation, the sensor body 302 was fixed to the measurement jig 401 to perform the evaluation. Here, the sensor body 302 was disposed such that one of the reinforcing members 324, 325 was positioned on an upper surface 402a (cf. FIG. 15A) of the first lever 402 and the other was positioned on an upper surface 403a (cf. FIG. 15A) of the second lever 403, and both ends of the sensor body 302 were fixed to the first lever 402 and the second lever 403, respectively, by using clamp members 407. In the present evaluation, the sensor body 302 was attached to the measurement jig 401 via a low-friction stretching cloth such that the sensor element 40 was substantially uniformly elongated over the entire length direction of the sensor element without being inhibited by the cylindrical member 405.

In the present evaluation, the state shown in FIGS. 14 and 15A is the initial state with the bending angle of 0°.

Note that the photograph of FIG. 14 and the schematic diagrams of FIGS. 15A and 15B are upside down.

Next, while the first lever 402 was fixed, the second lever 403 was rotated around the cylindrical member 405, and the angle at which the upper surface 403a of the second lever 403 at that time was rotated from the initial state was set to the bending angle.

In this case, as shown in FIG. 15B, the sensor body 302 is bent while being elongated.

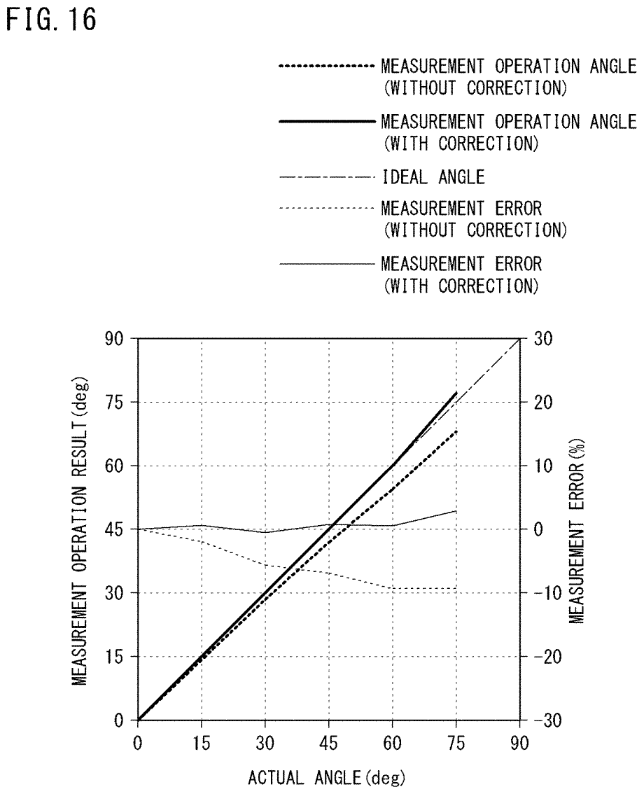

In the present evaluation, the second lever 403 was rotated every 15° to 75°, the difference between the length of the first reference line segment S1 provided in the intermediate layer 321 and the length of the second reference line segment S2 provided in the intermediate layer 321 was calculated based on the change in capacitance obtained from the detection portion 316 of the sensor element 40 provided in the sensor body, and the bending angle of the sensor body was calculated using the difference of the lengths. FIG. 16 shows the results. Here, as the length corresponding to the length of the first reference line segment S1 and the length corresponding to the length of the second reference line segment S2, the lengths in the longitudinal directions of the respective detection portions 316 of the two sensor elements were calculated.

In FIG. 16, (1) measurement operation angles (without correction) and (2) measurement operation angles (with correction) were each plotted and connected by a dotted line or a solid line. For each measurement operation angle, (3) a deviation from an actual bending angle (actual angle) (measurement error) was calculated and shown in FIG. 16. FIG. 16 further shows a theoretical value as an ideal angle.

(1) The measurement operation angle (without correction) is a value calculated based on the difference between the length of the first reference line segment S1 and the length of the second reference line segment S2 and the initial thickness of the intermediate layer 321 after the respective amounts of changes (amounts of elongation) in the lengths of the two reference line segments were calculated based on the voltages (voltages corresponding to the capacitances of the detection portions of the sensor elements 40) measured in the respective sensor elements 40.

That is, the measurement operation angle (without correction) was calculated using Equation (3) above, except that the angle unit was converted from “radian” to “°”.

(2) The measurement operation angle (with correction) is a value calculated based on the difference between the length of the first reference line segment S1 and the length of the second reference line segment S2 and the thickness of the intermediate layer 321 after the respective amounts of changes (amounts of elongation) in the lengths of the two reference line segments were calculated by the method of (1), the value being a value calculated by correcting the thickness.

Here, as the correction of the thickness, the initial thickness was first corrected to [(initial thickness of intermediate layer: 2 mm)+(thickness of sensor element: 0.4 mm)÷2×2].

Further, in order to correct the change in thickness that occurs at the time of the bending deformation, the bending angle was calculated using Equation (5) above. At this time, in practice, the bending angle was calculated using Equation (10) below in order to make the unit of the angle “°”.

$$\begin{matrix}[ Mathematical Expression 5 ] & \\ θ ( deg ) = \frac{{L}_{3 ( S 1 )} - {L}_{3 ( S 2 )}}{{t}_{0}} \times \sqrt{\frac{\frac{{L}_{3 ( S 1 )} + {L}_{3 ( S 2 )}}{2}}{{L}_{0}}} \times \frac{360}{2 π} & ( 10 )\end{matrix}$$

(3) The measurement error is a value obtained by calculating the “difference from the actual bending angle (actual angle)” as a percentage of the “actual bending angle” for each of (1) the measurement operation angle (without correction) and (2) the measurement operation angle (with correction).

As shown in FIG. 16, both (1) the measurement operation angle (without correction) and (2) the measurement operation angle (with correction) are substantially proportional to the actual bending angle (actual angle), so that it has become clear that the sensor body 302 according to the embodiment of the present invention can measure the bending angle.

In addition, in the measurement of bending accompanied with stretching, it became clear that (2) the measurement operation angle (with correction) is more excellent in measurement accuracy than (1) the measurement operation angle (without correction), because it is possible to detect the bending angle without obtaining a proportional coefficient by calibration or the like by simply including the actual dimension of the intermediate layer in the calculation formula.

<2. Bending Evaluation 2>

In the bending evaluation 1, the sensor body 302 was fixed to the measurement jig 401 in a state where the bending angle was 0°, and then the second lever 403 was rotated while the first lever 402 was fixed. Therefore, in the evaluation of the bending evaluation 1, the bending deformation occurs with elongation.

On the other hand, in the bending evaluation 2, the measurement of the bending angle at the time of occurrence of only the bending deformation without elongation was evaluated. The bending evaluation 2 was evaluated using the measurement jig 401 as in the bending evaluation 1.

Specifically, the actual bending angle (actual angle) between the upper surface 403a of the first lever 402 and the upper surface 402a of the second lever 403 provided in the measurement jig 401 was adjusted to any of 0°, 15°, 30°, 45°, 60°, 75°, and 90° in advance, the sensor body 302 was attached to the measurement jig 401 in this state by a similar method to that in the bending evaluation 1, and the capacitance (the voltage after CV conversion corresponding to the capacitance) of the detection portion 316 of the two sensor elements 40 provided in the sensor body 302 was measured. Note that the sensor body 302 was attached to the measurement jig 401 such that tensile stress was not applied.

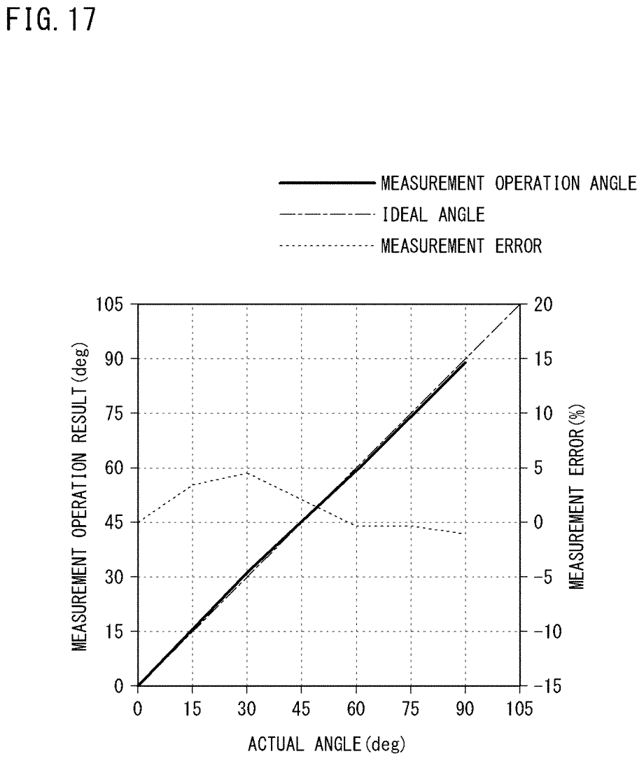

FIG. 17 shows a measurement operation angle calculated from the difference between the lengths in the longitudinal directions of the respective detection portions of the sensor element and the thickness of the intermediate layer by calculating the lengths of the detection portions from the voltages corresponding to the capacitances of the detection portions.

In the present evaluation as well, as in the bending evaluation 1, as the length corresponding to the length of the first reference line segment S1 and the length corresponding to the length of the second reference line segment S2, the lengths in the longitudinal directions of the respective detection portions 316 of the two sensor elements were calculated.

Here, the calculation of the measurement operation angle at each bending angle was performed twice, and in FIG. 17, the average values in the calculation performed twice were plotted, and the plots were connected by a solid line.

As shown in FIG. 17, the measurement operation angle, calculated based on the capacitances of the respective detection portions of the two sensor elements 40 provided in the sensor body 302, is substantially proportional to the bending angle (actual angle), and it has become clear that the sensor body 302 of the present invention can measure the bending angle.

In the present example, at the time of calculating a length Lb in the longitudinal direction of the detection portion from the voltage obtained in accordance with the capacitance of the detection portion, Equation (11) below was adopted.

Lb=La×(Vb/Va) (11)

(in the formula (11), Va is an output voltage measured by the sensor element in a non-deformed state, Vb is an output voltage measured by the sensor element in a deformed state, La is the length in the longitudinal direction of the detection portion in the sensor element in the non-deformed state, and Lb is the length in the longitudinal direction of the detection portion in the sensor element in the deformed state.)

<3. Elongation Evaluation>

Here, only the elongation deformation was caused to occur in the longitudinal direction (the X-direction of the sensor element) without accompanying the bending deformation in the sensor body 302, and the capacitances of the respective detection portions of the two sensor elements 40 provided in the sensor body 302 at that time were CV-converted, and then the average value of the output voltage was obtained.

At this time, the sensor body 302 was elongated to 20 mm in increments of 2 mm by gripping the reinforcing members 324, 325.

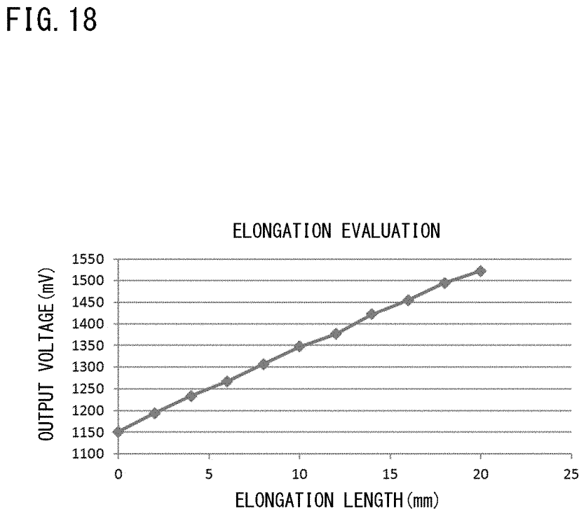

FIG. 18 shows the relationship between the average value of the output voltage and the elongation length (amount of elongation) of the sensor body.

As shown in FIG. 18, the average value of the output voltages corresponding to the capacitances of the respective detection portions of the two sensor elements 40 provided in the sensor body 302 is substantially proportional to the amount of elongation of the sensor body, and it has become clear that the sensor body according to the embodiment of the present invention can also measure the amount of elongation.

From the above results, it has become clear that the sensor body of the present invention can easily and accurately measure the amount of elongation and the bending angle of the measuring object.

### REFERENCE SIGNS LIST

1: SENSOR DEVICE

2, 102, 202, 302: SENSOR BODY

3: ANALYZER

4: INDICATOR

11: DIELECTRIC LAYER

12A: TOP ELECTRODE LAYER (FIRST ELECTRODE LAYER)

12B: BOTTOM ELECTRODE LAYER (SECOND ELECTRODE LAYER)

13A: TOP CONDUCTING WIRE

13B: BOTTOM CONDUCTING WIRE

14A: TOP CONNECTING PORTION

14B: BOTTOM CONNECTING PORTION

15A, 45A: TOP PROTECTIVE LAYER

15B, 45B: BOTTOM PROTECTIVE LAYER

16, 316: DETECTION PORTION

19, 319: LEAD WIRE

21, 121, 321: INTERMEDIATE LAYER

22, 40, 122: Sensor element

22A, 122A: first sensor element

22B, 122B: SECOND SENSOR ELEMENT

23A, 23B: ATTACHMENT MEMBER

41A: FIRST DIELECTRIC LAYER

41B: SECOND DIELECTRIC LAYER

42A: FIRST ELECTRODE LAYER

42B: SECOND ELECTRODE LAYER

42C: THIRD ELECTRODE LAYER

43A: FIRST CONDUCTING WIRE

43B: SECOND CONDUCTING WIRE

43C: THIRD CONDUCTING WIRE

44A: FIRST CONNECTING PORTION

44B: SECOND CONNECTING PORTION

44C: THIRD CONNECTING PORTION

124, 125, 324, 325: REINFORCING MEMBER

401: MEASUREMENT JIG

S1, S11: FIRST REFERENCE LINE SEGMENT

S2, S12: SECOND REFERENCE LINE SEGMENT

P1a, P1b, P11a, P11b: END POINT

RS1: FIRST REFERENCE PLANE

RS2: SECOND REFERENCE PLANE

1. A sensor body comprising:
a plate-like flexible intermediate layer that is stretchable in a surface direction;
a first sensor element laminated on one surface of the intermediate layer; and
a second sensor element laminated on the other surface of the intermediate layer facing the one surface,
wherein
the first sensor element is configured to be capable of detecting a length corresponding to a length of a first reference line segment on the one surface of the intermediate layer, and
the second sensor element is configured to be capable of detecting a length corresponding to a length of a second reference line segment that is on the other surface of the intermediate layer and parallel to the first reference line segment.

2. The sensor body according to claim 1, wherein
the first sensor element and the second sensor element are both capacitive sensor elements,
each of the capacitive sensor elements includes
a dielectric layer made of an elastomer,
a first electrode layer formed on an upper surface of the dielectric layer, and
a second electrode layer formed on a lower surface of the dielectric layer, each of the capacitive sensor elements having a part in which the first electrode layer and the second electrode layer face each other, the part serving as a detection portion, and
a capacitance of the detection portion changes in accordance with deformation of the dielectric layer.

3. The sensor body according to claim 1, further comprising a plate-like attachment member having the same thickness as the intermediate layer and higher rigidity than the intermediate layer,
wherein the attachment member is provided outside each end of the first reference line segment of the intermediate layer.

4. The sensor body according to claim 1, used for measuring a joint angle of a human body.

5. A sensor device comprising:
the sensor body according to claim 1; and
an analyzer,
wherein
the analyzer measures a change in the length corresponding to the length of the first reference line segment and a change in the length corresponding to the length of the second reference line segment at a time of deformation of the sensor body and calculates a bending angle and amount of elongation of a measuring object based on the obtained measurement results.

6. The sensor device according to claim 5, wherein the first sensor element and the second sensor element of the sensor body are both capacitive sensor elements,
each of the capacitive sensor elements includes
a dielectric layer made of an elastomer,
a first electrode layer formed on an upper surface of the dielectric layer, and
a second electrode layer formed on a lower surface of the dielectric layer, each of the capacitive sensor elements having a part in which the first electrode layer and the second electrode layer face each other, the part serving as a detection portion, and
a capacitance of the detection portion changes in accordance with deformation of the dielectric layer.

7. The sensor device according to claim 5, wherein the sensor body further comprises a plate-like attachment member having the same thickness as the intermediate layer and higher rigidity than the intermediate layer,
wherein the attachment member is provided outside each end of the first reference line segment of the intermediate layer.

8. The sensor device according to claim 5, wherein the sensor body is used for measuring a joint angle of a human body.
